(19) United States
(12) Patent Application Publication
(10) Pub. No.: US 2024/0374897 A1
(43) Pub. Date: Nov. 14, 2024

(54) Closed-Loop Neuromodulation for the Treatment of Affective Neuropsychiatric Disorders

(71) Applicant: The Regents of the University of California, Oakland, CA (US)

(72) Inventors: Katherine Scangos, San Francisco, CA (US); Andrew Krystal, San Francisco, CA (US); Edward Chang, San Francisco, CA (US)

(21) Appl. No.: 18/287,094

(22) PCT Filed: May 3, 2022
(86) PCT No.: PCT/US2022/027429

§ 371 Date: Oct. 16, 2023

Related U.S. Application Data
(60) Provisional application No. 63/232,857, filed on Aug. 13, 2021.
(60) Provisional application No. 63/183,833, filed on May 4, 2021.

(51) Int. Cl. A61N 1/36; A61N 1/05; A61N 1/372

(52) U.S. Cl. CPC A61N 1/36096; A61N 1/36139; A61N 1/36157; A61N 1/37247; A61N 1/0529

(57) ABSTRACT

The present disclosure provides methods and systems for treating affective disorders such as depression and/or anxiety and/or related disorders. Closed-loop therapy is performed with a neurostimulator that records electroencephalographic signals from neural activity associated with symptoms of a neuropsychiatric disorder and delivers electrical stimulation when pre-specified patterns of neural activity associated with a symptom are detected. Methods are also disclosed for mapping the brain of a patient to optimize placement of electrodes for detecting neural activity associated with symptoms of a neuropsychiatric disorder and to optimize placement of electrodes for delivery of electrical stimulation. Methods of monitoring and evaluating the effects of electrical stimulation on clinical symptoms and status are also provided.

### CROSS-REFERENCE

This application claims the benefit of U.S. Provisional Patent Application No. 63/183,833, filed May 4, 2021, and U.S. Provisional Patent Application No. 63/232,857, filed Aug. 13, 2021, which applications are incorporated herein by reference in their entirety.

### INTRODUCTION

The development of novel effective treatments for neuropsychiatric disorders is a leading goal of 21^{st}-century medicine. Millions of individuals worldwide suffer from Major Depressive Disorder (MDD), Generalized Anxiety Disorder (GAD), Post-Traumatic Stress Disorder (PTSD), Addiction, Anorexia, Obsessive-Compulsive Disorder (OCD), Bipolar Disorder (BD), Chronic Pain, and related conditions. Vast numbers of patients remain ineffectively treated. Accordingly, neuropsychiatric disorders constitute the largest worldwide disease burden of all clinical categories, accounting for 22.7% of all years lived with disability.

Established treatment modalities—including pharmacological treatment, cognitive behavioral therapy (CBT), surgical lesioning and electroconvulsive therapy (ECT)-provide lasting benefits to a proportion of individuals but prove inadequate for many. For example, it is estimated that in the case of MDD, ˜40-50% of adult patients undergoing treatment with selective serotonin reuptake inhibitors fail to respond. While CBT has shown some effectiveness, for example in preventing MDD relapse following pharmacologic treatment, relapse rates remain significant (˜35-50% in long-term studies). Surgical lesioning approaches for neuropsychiatric disorders are highly invasive and do not yield optimal outcomes (e.g., full response rates of <60% in the case of anterior capsulotomy for OCD). No fully reliable treatment method exists for any of the major affective disorders.

Gross-level electrophysiological intervention in the form of ECT has been long used for some neuropsychiatric disorders, but many severely affected patients experience no lasting relief with ECT and endure significant unwanted cognitive side-effects. Transcranial stimulation methods, while non-invasive, have not provided conclusive evidence regarding their effectiveness—findings are perhaps strongest for MDD, but in that case response rates in the short term are still typically <60%. More advanced methods involving targeted direct stimulation of specific subregions of the brain, such as subgenual cingulate, have provided promising early results in recent years but have failed in clinical trials owing to variability in patient response.

### SUMMARY

Methods and systems for treatment of a neuropsychiatric disorder in a subject are provided. Closed-loop therapy is performed with a neurostimulator that records electroencephalographic signals from neural activity associated with a symptom of a neuropsychiatric disorder and delivers electrical stimulation when pre-specified patterns of neural activity associated with the symptom of the neuropsychiatric disorder are detected. Methods are also disclosed for mapping the brain of a patient to optimize placement of electrodes for detecting neural activity associated with symptoms of a neuropsychiatric disorder and to optimize placement of electrodes for delivery of electrical stimulation. Methods of monitoring and evaluating the effects of electrical stimulation on clinical symptoms and status are also provided. The methods and systems can be used for treatment of affective disorders related to anxiety and/or depression and/or related disorders, including, without limitation, Major Depressive Disorder (MDD), Generalized Anxiety Disorder (GAD), Post-Traumatic Stress Disorder (PTSD), Addiction, Anorexia. Obsessive-Compulsive Disorder (OCD), and Bipolar Disorder (BD), and Chronic Pain.

In certain cases, the neuropsychiatric disorder may be depression and applying the electrical stimulation treats depression. In certain cases, the neuropsychiatric disorder may be anxiety and applying the electrical stimulation treats anxiety. The neuropsychiatric disorder may be one or more of Major Depressive Disorder (MDD), Generalized Anxiety Disorder (GAD), Post-Traumatic Stress Disorder (PTSD), Addiction, Anorexia, Obsessive-Compulsive Disorder (OCD), Bipolar Disorder (BD) or Chronic Pain.

In one aspect, a closed-loop method for treating a neuropsychiatric disorder in a subject is provided, the method comprising: mapping a brain of a subject to identify an optimal location in a first brain region for positioning a first electrode for delivery of electrical stimulation to treat the neuropsychiatric disorder; mapping the brain of the subject to identify an optimal location in a second brain region for positioning a second electrode for detection of an electroencephalographic signal from neural activity associated with a symptom of the neuropsychiatric disorder; positioning the first electrode at the optimal location in the first brain region to deliver electrical stimulation to the first brain region of the subject; positioning the second electrode at the optimal location in the second brain region to detect the electroencephalographic signal from the neural activity associated with the symptom of the neuropsychiatric disorder; detecting the electroencephalographic signal at the second region of the brain of the subject using the second electrode; and applying electrical stimulation to the first brain region using the first electrode in a manner effective to treat the neuropsychiatric disorder in the subject when the electroencephalographic signal that is detected using the second electrode exceeds a threshold level. In some embodiments, the first brain region and the second brain region are the same. In other embodiments, the first brain region and the second brain region are different.

In certain embodiments, the method further comprises using a control algorithm to automate said applying electrical stimulation when the electroencephalographic signal exceeds the threshold level. In some embodiments, the control algorithm comprises a machine learning algorithm such as a supervised machine learning algorithm. In some embodiments, the control algorithm further modulates one or more programmed stimulation parameters based on the level of the electroencephalographic signal.

In certain embodiments, the method further comprises positioning a plurality of electrodes at the second brain region for detection of the electroencephalographic signal by stereoelectroencephalography (sEEG).

In certain embodiments, the first brain region comprises a ventral capsule/ventral striatum (VC/VS) region, a subgenual cingulate (SGC) region, or an orbitofrontal cortex (OFC) region, or other region of the brain. The site chosen for stimulation may differ for different subjects and will depend on the mapping of the brain of an individual subject to identify the optimal location for positioning an electrode for delivery of electrical stimulation to treat the neuropsychiatric disorder.

In certain embodiments, the second brain region comprises a right amygdala region, a left amygdala region, a right orbitofrontal cortex region, a left subgenual cingulate region, or a right hippocampus region, or other region of the brain. The site chosen for detection may differ for different subjects and will depend on the mapping of the brain of an individual subject to identify the optimal location for positioning an electrode for detecting electroencephalographic signals from neural activity associated with a symptom of the neuropsychiatric disorder.

In certain embodiments, the electroencephalographic signal is gamma frequency power.

The method may further include assessing effectiveness of the treatment in the subject. For example, the subject may be monitored for a period of time, wherein increasing numbers of the electroencephalographic signals exceeding the threshold level detected by the second electrode within a later recording period compared to an earlier recording period indicate increasing severity of the symptom of the neuropsychiatric disorder; and decreasing numbers of electroencephalographic signals exceeding the threshold level detected by the second electrode within a later recording period compared to an earlier recording period indicate decreasing severity of the symptom of the neuropsychiatric disorder. In some embodiments, the subject is monitored continuously or intermittently.

Aspects of the methods include applying the electrical stimulation unilaterally or bilaterally. In some embodiments, the electrical stimulation is applied at least two times, wherein the site to which the electrical stimulation is applied is alternated or otherwise spatially or temporally patterned.

Also provided is a method for ameliorating a symptom of a neuropsychiatric disorder in a subject. In one aspect, a closed-loop method for ameliorating a symptom of a neuropsychiatric disorder in a subject is provided, the method comprising: mapping a brain of a subject to identify an optimal location in a first brain region for positioning a first electrode for delivery of electrical stimulation to ameliorate the symptom of the neuropsychiatric disorder; mapping the brain of the subject to identify an optimal location in a second brain region for positioning a second electrode for detection of an electroencephalographic signal from neural activity associated with the symptom of the neuropsychiatric disorder; positioning the first electrode at the optimal location in the first brain region to deliver electrical stimulation to the first brain region of the subject; positioning the second electrode at the optimal location in the second brain region to detect the electroencephalographic signal from the neural activity associated with the symptom of the neuropsychiatric disorder; detecting the electroencephalographic signal at the second region of the brain of the subject using the second electrode; and applying electrical stimulation to the first brain region using the first electrode in a manner effective to ameliorate the symptom of the neuropsychiatric disorder in the subject when the electroencephalographic signal that is detected using the second electrode exceeds a threshold level. In some embodiments, the first brain region and the second brain region are the same. In other embodiments, the first brain region and the second brain region are different.

The symptom may be depression, wherein applying the electrical stimulation ameliorates depression. In some cases, depression may be measured by using the Visual Analogue Scale for Depression (VAS-D), Hamilton Depression Rating Scale (HAM-D), Montgomery-Asberg Depression Rating Scale (MADRS), or Beck Depression Inventories (BDI) score, wherein applying the electrical stimulation is effective in reducing severity of depression according to the VAS, HAM-D, MADRS, or BDI score

In some cases, the symptom may be anxiety, wherein applying the electrical stimulation ameliorates anxiety. In some cases, anxiety may be measured by Visual Analogue Scale for Anxiety (VAS-A) or Beck Anxiety Inventories (BAI) score, wherein applying the electrical stimulation is effective in reducing anxiety according to the VAS-A or the BAI score.

In some cases, the symptom may be chronic-pain-related distress, wherein applying the electrical stimulation ameliorates chronic-pain-related distress. In some cases, chronic-pain-related distress may be measured by a Visual Analog Scale, wherein applying the electrical stimulation may be effective in reducing the Visual Analog Scale score.

The stimulation electrode may be a brain-penetrating electrode or a non-brain penetrating electrode. The detection electrode may be a brain-penetrating electrode or a non-brain penetrating electrode. Electrical stimulation may be applied as summarized above.

In certain aspects, an electrical signal is measured from the brain to determine severity of a symptom of a neuropsychiatric disorder in a subject by intracranial electroencephalography (iEEG) or a similar technique. In some cases, the electrical signal may be measured continuously, intermittently, or both. For continuous measurement, electroencephalography may be performed for an uninterrupted period of 10 sec-24 hrs or more, e.g., 30 sec-18 hrs, 1 min-12 hrs, 10 min-8 hrs, 30 sec-10 min, 1 min-10 min, 3 min-10 min, or 3 min-6 min. In some cases, a symptom of a neuropsychiatric disorder may be detected by the presence of increased power of gamma (30 Hz to 200 Hz) frequency electrical activity in the brain. In some cases, treatment of the neuropsychiatric disorder may suppress symptoms and concomitant increases in power of gamma (30 Hz to 200 Hz) frequency electrical activity in the brain. The decrease in gamma frequency power measured from the brain may be a decrease of 5%-20%, or more, such as, at least 5%, at least 10%, at least 15%, at least 20%, at least 40%, or more as compared to that prior to the treatment. The electrical activity may be recorded from any region of the brain, such as the right amygdala, left amygdala, right orbitofrontal cortex, left subgenual cingulate, and/or right hippocampus region, and/or regions of the brain in neural connection with the amygdala. In some cases, the electrical activity may be recorded from multiple locations in the brain and activity within different frequency bands averaged. For example, frequencies in the 30 Hz to 200 Hz range, recorded from multiple locations in the brain, may be averaged. In some cases, the multiple locations may include multiple locations in the right amygdala, left amygdala, right orbitofrontal cortex, left subgenual cingulate, and/or right hippocampus. The electrical activity may be measured prior to, during, and/or after stimulation of the brain as disclosed herein. In some cases, an increase in cortical excitability may be indicative of efficacious treatment. In some cases, other neural features, from a single brain region or a combination of brain regions, may be associated with a symptom of a neuropsychiatric disorder, or may be indicative of efficacious treatment.

In some cases, the brain activity, measured from the brain (e.g., gamma frequency electroencephalographic signals) after a subject having a neuropsychiatric disorder is treated according to the methods and systems of the present disclosure, may be below a threshold brain activity (threshold level of gamma frequency electroencephalographic signals). In certain cases, the threshold brain activity may be the brain activity known to be associated with the neuropsychiatric disorder. In some cases, gamma frequency electroencephalographic signals, measured from the brain after a subject having a neuropsychiatric disorder is treated according to the methods and systems of the present disclosure, may be below a threshold level.

In some cases, measurement of electrical signals from the brain may be combined with an alternate method for assessing the neuropsychiatric disorder. For example, the neuropsychiatric disorder may be assessed by mood assessment tools. Mood assessment tools may include verbal mood report or immediate mood scaler.

In certain embodiments, a closed-loop method for treating major depressive disorder (MDD) in a subject is provided, the method comprising: positioning a first electrode to deliver electrical stimulation to a first region of the brain of the subject; positioning a second electrode to detect a gamma frequency electroencephalographic signal from neural activity associated with MDD symptoms at a second region of the brain of the subject; detecting the gamma frequency electroencephalographic signal using the second electrode; and applying electrical stimulation to the first region of the brain of the subject using the first electrode in a manner effective to treat the MDD symptoms in the subject when the gamma frequency electroencephalographic signal that is detected using the second electrode exceeds a threshold level. In some embodiments, bipolar electrical stimulation is applied at the first region of the brain of the subject. In some embodiments, electrical stimulation is applied at a current ranging from 1 mA to 6 mA, including any current within this range such as 1, 1.5, 2, 2.5, 3, 3.5, 4, 4.5, 5, 5.5, or 6 mA. In some embodiments, electrical stimulation is applied for a time ranging from 5 seconds to 10 minutes a day, including any amount of time in between such as 5 seconds, 10 seconds, 20 seconds, 30 seconds, 40 seconds, 50 seconds, 1 minute, 2 minutes, 3 minutes, 4 minutes, 5 minutes, 6 minutes, 7 minutes, 8 minutes, 9 minutes, or 10 minutes a day. In other embodiments, electrical stimulation is applied continuously for a time ranging from 1 hour a day to 24 hours a day, including any amount of time within this range such as 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, or 24 hours a day.

In certain embodiments, the first brain region comprises a ventral capsule/ventral striatum (VC/VS) region, a subgenual cingulate (SGC) region, or an orbitofrontal cortex (OFC) region, or other region of the brain. The site chosen for stimulation may differ for different subjects and will depend on the mapping of the brain of an individual subject to identify the optimal location for positioning an electrode for delivery of electrical stimulation to treat MDD.

In certain embodiments, the second brain region comprises a right amygdala region, a left amygdala region, a right orbitofrontal cortex region, a left subgenual cingulate region, or a right hippocampus region, or other region of the brain. The site chosen for detection may differ for different subjects and will depend on the mapping of the brain of an individual subject to identify the optimal location for positioning an electrode for detecting electroencephalographic signals from neural activity associated with a symptom of MDD.

In certain embodiments, the method further comprises monitoring the subject for a period of time, wherein increasing numbers of gamma frequency electroencephalographic signals exceeding the threshold level detected by the second electrode within a later recording period compared to an earlier recording period indicate increasing severity of MDD symptoms; and decreasing numbers of gamma frequency electroencephalographic signals exceeding the threshold level detected by the second electrode within a later recording period compared to an earlier recording period indicate decreasing severity of MDD symptoms. For example, the subject can be monitored continuously or intermittently.

In another aspect, a system for treating a neuropsychiatric disorder in a subject is provided, the system comprising: a stimulation electrode adapted for positioning at a first region of the brain of the subject; a detection electrode adapted for positioning at a second region of the brain of the subject and for recording an electroencephalographic signal from the second region of the brain of the subject; and a processor programmed to instruct the stimulation electrode to apply an electrical stimulation to the first region of the brain in a manner effective to treat the neuropsychiatric disorder in the subject when an electroencephalographic signal that exceeds a threshold level is detected using the second electrode.

In certain embodiments, the processor is further programmed to modulate one or more programmed stimulation parameters according to the algorithm's control law; and apply the modulated electrical stimulation to the first region of the brain via the stimulation electrode in a manner effective to treat the neuropsychiatric disorder.

In certain embodiments, the first brain region comprises a ventral capsule/ventral striatum (VC/VS) region, a subgenual cingulate (SGC) region, an orbitofrontal cortex (OFC) region, or other region of the brain.

In certain embodiments, the second brain region comprises a right amygdala region, a left amygdala region, a right orbitofrontal cortex region, a left subgenual cingulate region, or a right hippocampus region, or other region of the brain.

In certain embodiments, the processor is further programmed to set a maximum number of electrical stimulations per day.

In certain embodiments, the processor is further programmed to set a total amount of time of electrical stimulation per day.

### BRIEF DESCRIPTION OF THE DRAWINGS

FIGS. 1A-ID. Neural Biomarker of a Severely Depressed State. FIG. 1A. Overall approach for biomarker detection. Ten intracranial electrodes were implanted across the corticolimbic circuit for 10 days. Resting-state neural activity was recorded for 15-minutes and paired with depression clinical reports. FIG. 1B. Clustering of clinical reports on dimensions of anxiety and depression resulted in 2 symptom states. Shading added to aid visualization. FIGS. 1C-1D. Top neural features (defined by F-score, ANOVA) that discriminated high vs. low symptom severity states. AUC curves show predictive test performance of logistic regression classifier fit with these 6 features in comparison to a null model. Models with two levels of spatial resolution are shown (brain region level model: c, electrode contact level model: FIG. 1D.

FIGS. 2A-2E. Identification of Limbic Subnetwork. FIG. 2A. Directed network graph of right hemisphere where percent circumference indicates strength of the connection between any two brain regions. Color indicates the starting location for each set of connections. FIG. 2B. Connectivity strength from the network graph is quantified by calculating the weighted indegree and outdegree, which indicate brain regions that are influential hubs in the corticolimbic network. VC/VS has the highest weighted outdegree suggesting it has strong connections to other brain regions as observed in a AMY and HPC have the highest weighted indegree, suggesting they receive connections from many other brain regions. FIG. 2C. Example of dose dependent mean EPs (left). Summary of EPs (z-scored relative to baseline) across the corticolimbic network due to single pulse stimulation in the right VC/VS is shown overlayed on brain as heatmap (right). Warmer colors indicate a larger N1 amplitude, suggesting VC/VS-AMY is a tightly connected subnetwork. FIG. 2D. Location of right sided iEEG leads with fiber tracts (color coded by orientation) showing structural connections between AMY and the clinically responsive VC/VS electrodes, from deterministic tractography. Lead location indicated by color as in a-b. FIG. 2E. Change in gamma biomarker following a period of continuous stimulation for those trials that led to a reduction in symptom severity (red), and those that did not reduce symptom severity (blue).

FIGS. 3A-3J. Implementation of Closed-loop Neuromodulation. FIG. 3A. Illustration of fully implantable deep brain stimulation and recording device. FIG. 3B. Reproducibility of targeting showing robust engagement of the stria-terminalis and ansa-peduncularis. VC/VS lead: yellow; AMY lead: pink. FIG. 3C. Distribution of symptom severity scores in relation to clusters identified in the mapping study for trials with 10 minutes of resting state continuous neural recordings collected for the purpose of biomarker identification. FIG. 3D. Positive correlation between gamma power in AMY contacts ½, ¾ within the 10-minute trials, and HAMD-6 score. FIG. 3E. Reproducibility of clinical effects. Each point represents a stimulation trial (n=6, 2, 4, 5 for VAS-D; n=6, 2, 3, 4 for HAMD) at different bipolar configurations across the right VC/VS and AMY. FIG. 3F. Left: Effect of burst duration on clinical measures. Stimulation parameters: contact 3+/4−, 1 mA, 36 s total stimulation across 15.6 min. in 6-36 s intervals. Highlighted condition (6 s burst duration) selected for implementation of closed-loop therapy. Right: Effect of increasing dose (1 to 2 mA) and changing contacts (3+/4− to 2+/3−) on clinical measures. Faded colors indicate that ON/OFF states were detected by patient (1 trial per condition). FIG. 3G. Schematic of closed-loop control for the deep brain stimulation device. Signals are recorded continuously. If the neural biomarker is detected, indicating a highly depressed state, a pulse of stimulation is delivered. FIG. 3H. Positive correlation between number of gamma detections by NeuroPace RNS System within the same 10-minute trials as in f) and HAMD-6 score. FIG. 3I. Symptom severity scores in the week pre vs. post the onset of closed-loop stimulation (n=3, 31 for VAS-D, n=2, 30 for HAMD6). FIG. 3J. The relationship between daily mood ratings (purple) and number of daily biomarker detections (gray) over two months of treatment with the RNS System. Dotted lines indicate dynamic time warping (DTW)-computed distance between VAS-D scores and daily biomarker detection numbers normalized to their means (left). Significance was assessed by comparing the DTW-distance to the distance computed from 10,000 randomly scrambled biomarker time series (right).

FIGS. 4A-4B. Network Connectivity. FIG. 4A. Evoked potentials (z-scored relative to baseline) across the corticolimbic network due to single pulse stimulation in the right VC/VS, OFC and SGC is shown overlayed on brain as heatmap. Warmer colors indicate a larger N1 amplitude. FIG. 4B. Location of right sided sEEG leads targeting AMY (pink), VC/VS (orange), SGC (green), and OFC (blue). Fiber tracts (color coded by orientation) show putative structural connections between candidate pairs of stimulation and sensing contacts (VC/VS-AMY, VC/VS-OFC, SGC-AMY. OFC-AMY) from deterministic tractography using 3 mm ROIs centered on each contact. Tractography parameters were the same for all pairs: minimum FA=0.1; minimum fiber length=80 mm; maximum angulation=20 degrees.

FIG. 5. Overall Approach to Stimulation and Sensing Target Selection. Multimodal method for personalized responsive stimulation multi-lead targeting began with clinical mapping to identify candidate sites where stimulation reliably led to symptom improvement across a range of doses and symptom severity states. Candidate sensing locations were identified by pairing resting state neural activity with symptom severity ratings to identify spectral power biomarkers that correlated with depression. The relationship between candidate stimulation and sensing targets were then tested using three approaches. First, effective network connectivity was assessed by examining the evoked response at nodes across the network due to single pulse stimulation at candidate targets. Second, structural connectivity between candidate contact pairs was assessed using tractography. Influential tracts were identified to help with retargeting during implantation of chronic stimulation device. Finally, the feasibility of closed-loop control was assessed by examining the effect of stimulation in candidate stimulation sites on putative biomarkers identified in candidate sensing locations. This personalized approach enabled us to identify one best stimulation and sensing target pair which were then utilized for the implantation of the RNS System and delivery of closed-loop neurostimulation therapy.

FIG. 6. Flowchart of discovery strategy for development of personalized closed-loop neurostimulation therapy.

### DETAILED DESCRIPTION

Methods and systems for treatment of a neuropsychiatric disorder in a subject are provided. Closed-loop therapy is performed with a neurostimulator that records electroencephalographic signals from neural activity associated with a symptom of a neuropsychiatric disorder and delivers electrical stimulation when pre-specified patterns of neural activity associated with the symptom of the neuropsychiatric disorder are detected.

Neuropsychiatric disorders that can be treated with the systems and methods disclosed herein may include, without limitation, MDD, GAD, PTSD, Addiction, OCD, Anorexia Nervosa, Bipolar Disorder, Chronic Pain, and related conditions. A patient may suffer from one or more of these neuropsychiatric disorders. The methods disclosed herein may treat more than one neuropsychiatric disorder in the same patient.

Before exemplary embodiments of the present invention are described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and exemplary methods and materials may now be described. Any and all publications mentioned herein are incorporated herein by reference to disclose and describe the methods and/or materials in connection with which the publications are cited. It is understood that the present disclosure supersedes any disclosure of an incorporated publication to the extent there is a contradiction.

It must be noted that as used herein and in the appended claims, the singular forms “a”, “an”, and “the” include plural referents unless the context clearly dictates otherwise. Thus, for example, reference to “an electrode” or “the electrode” includes a plurality of such electrodes and reference to “a symptom” or “the symptom” includes reference to one or more symptoms, and so forth.

It is further noted that the claims may be drafted to exclude any element which may be optional. As such, this statement is intended to serve as antecedent basis for use of such exclusive terminology as “solely”, “only” and the like in connection with the recitation of claim elements, or the use of a “negative” limitation.

The publications discussed herein are provided solely for their disclosure prior to the filing date of the present application, Nothing herein is to be construed as an admission that the present invention is not entitled to antedate such publication by virtue of prior invention. Further, the dates of publication provided may be different from the actual publication dates which may need to be independently confirmed. To the extent such publications may set out definitions of a term that conflicts with the explicit or implicit definition of the present disclosure, the definition of the present disclosure controls.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

### Definitions

The term “neuropsychiatric disorders” is used herein to refer to a group of conditions that affect mood and/or behavior of a person suffering from the disorder. Neuropsychiatric disorders include Major Depressive Disorder (MDD), Generalized Anxiety Disorder (GAD), Post-Traumatic Stress Disorder (PTSD), Addiction, Anorexia, Obsessive-Compulsive Disorder (OCD), Bipolar Disorder (BD) and chronic pain.

The term “affective disorders” is used herein to refer to a group of neuropsychiatric disorders that typically affect mood of the person suffering from such a disorder. Neuropsychiatric disorders that affect the mood are also referred to as mood disorders and are commonly associated with depression and/or anxiety. The main types of affective disorders are depression, bipolar disorder, and anxiety disorder. Symptoms vary by individual, but they typically affect mood. They can range from mild to severe.

As used herein the term “depression” refers to a mental state of morbid sadness, dejection, or melancholy.

As used herein the term “anxiety” refers to an uncomfortable and unjustified sense of apprehension that may be diffuse and unfocused and is often accompanied by physiological symptoms.

As used herein the term “bipolar disorder” refers to a type of affective disorder in which the person suffering from this disorder goes through periods of depression and periods of mania (feeling extremely positive and active).

As used herein the term “anxiety disorder” refers to a neuropsychiatric disorder characterized by feelings of nervousness, anxiety, and even fear. Anxiety disorders include social anxiety (anxiety caused by social situations), post-traumatic stress disorder (anxiety, fear, and flashbacks caused by a traumatic event), generalized anxiety disorder (anxiousness and fear in general, with no particular cause), panic disorder (anxiety that causes panic attacks), and obsessive-compulsive disorder (obsessive thoughts that cause anxiety and compulsive actions).

By “treatment” or “treating” is meant that at least an amelioration of one or more symptoms associated with the condition afflicting the subject is achieved such that the patient has a desired or beneficial clinical result, where amelioration refers to at least a reduction in the magnitude of a parameter, e.g., a symptom, associated with the condition being treated. As such, treatment includes a broad spectrum of situations ranging from lessening intensity, duration or extent of impairment caused by a condition and/or correlated with a condition, up to and including completely eliminating the condition, along with any associated symptoms. Treatment therefore includes situations where the condition, or at least a symptom associated therewith, is completely inhibited, e.g., prevented from happening, or stopped, e.g., terminated, such that the subject no longer suffers from the condition, or at least the symptoms that characterize the condition. Treatment also includes situations where the progression of the condition, or at least the progression of a symptom associated therewith, is slowed, delayed, or halted. In such cases, a subject might still have residual symptoms associated the pathological condition, but any increase in the severity or magnitude of the symptoms is slowed, delayed, or prevented.

As used herein, the term “orbitofrontal cortex” or “OFC” refers to the defined area of the brain as known by one of skill in the art, as well as the surrounding or adjacent white matter tracts leading to and from OFC. OFC is a prefrontal cortex region in the frontal lobes in the brain. In humans it consists of Brodmann area 10, 11 and 47.

The term “subject” as used herein refers to a patient in need of the treatments disclosed herein. The patient may be a mammal, such as, a rodent, a feline, a canine, a primate, or a human, e.g., a child, an adolescent, an adult, such as a young, middle-aged, or elderly human. The patient may have been diagnosed as having a neuropsychiatric disorder, may be suspected of suffering from a neuropsychiatric disorder, or may be at risk of developing a neuropsychiatric disorder.

The term “user” as used herein refers to a person that interacts with a device and/system disclosed herein for performing one or more steps of the presently disclosed methods. The user may be the patient receiving treatment. The user may be a health care practitioner, such as, the patient's physician.

The term “symptom” as used in the context of a neuropsychiatric disorder, such as a symptom of a neuropsychiatric disorder refers to symptoms such as, anxiety, depression, fear, mania, and/or uncontrollable behavior.

### Methods

The present disclosure provides methods for treating a neuropsychiatric disorder in a subject. Also provided herein are methods for ameliorating a symptom of a neuropsychiatric disorder in a subject. Various steps and aspects of the methods will now be described in greater detail below.

Methods of the present disclosure utilize closed-loop therapy performed with a neurostimulator that records electroencephalographic signals from neural activity associated with a symptom of a neuropsychiatric disorder and delivers electrical stimulation when pre-specified patterns of neural activity associated with the symptom of the neuropsychiatric disorder are detected.

The method includes positioning a first electrode to deliver electrical stimulation (i.e., stimulation electrode) to a first brain region of the subject and positioning a second electrode to detect an electroencephalographic signal from neural activity associated with a symptom of the neuropsychiatric disorder (i.e., detection electrode) at a second brain region of the subject. One or more stimulation or detection electrodes may be positioned at the first brain region or the second brain region, respectively. In some embodiments, the first brain region and the second brain region are the same. In other embodiments, the first brain region and the second brain region are different. The stimulation and detection electrodes may be non-brain penetrating surface electrodes or brain-penetrating depth electrodes. The electrical stimulation may be applied to the first brain region using the stimulation electrode in a manner effective for treating the neuropsychiatric disorder and/or ameliorating a symptom of the neuropsychiatric disorder when an electroencephalographic signal is detected from the second brain region using the detection electrode that has a signal exceeding a threshold level indicating that the patient has a level of symptom severity in need of treatment. Such symptoms may include one or more of anxiety, depression, compulsive behavior, and the like.

The electrical stimulation may be applied using a single electrode, electrode pairs, or an electrode array. Electrical stimulation may be applied unilaterally or bilaterally to the first brain region. Bilateral electrical stimulation may be simultaneous or sequential. In some embodiments, the electrical stimulation is applied to more than one site. The site to which the electrical stimulation is applied may be alternated or otherwise spatially or temporally patterned.

Positioning an electrode for applying electrical stimulation at specified region(s) of the brain may be carried out using standard surgical procedures for placement of intra-cranial electrodes. As used herein, the phrases “an electrode” or “the electrode” refer to a single electrode or multiple electrodes such as an electrode array. As used herein, the term “contact” as used in the context of an electrode in contact with a region of the brain refers to a physical association between the electrode and the region. In other words, an electrode that is in contact with a region of the brain is physically touching the region of the brain. An electrode in contact with a region of the brain can conduct electricity into the brain. Electrodes used in the methods disclosed herein may be monopolar (cathode or anode) or bipolar (e.g., having an anode and a cathode). The electrode(s) used for applying electric stimulation to the specified region(s) of the brain are also referred to as stimulation electrode(s).

In certain embodiments, the brain region to which electrical stimulation is applied comprises the ventral capsule/ventral striatum (VC/VS) region, subgenual cingulate (SGC) region, or orbitofrontal cortex (OFC) region, or other regions of the brain suitable for stimulation. The site chosen for stimulation may differ for different subjects and will depend on mapping of the brain of an individual subject to identify the optimal location for positioning an electrode for delivery of electrical stimulation to treat a neuropsychiatric disorder, as discussed further below.

In certain cases, placing the electrode at the VC/VS, SGC, or OFC, or other region may involve positioning the electrode on the surface of the specified region(s) of the brain. Electrodes may be placed on the surface of the brain at the VC/VS, for example, at the nucleus accumbens (NAc) or the anterior limb of the internal capsule (ALIC), or both. The electrode may contact at least a portion of the surface of the brain at the VC/VS, SGC, or OFC, or other region. In some embodiments, the electrode may contact substantially the entire surface area at the VC/VS, SGC, or OFC, or other region. In some embodiments, the electrode may additionally contact area(s) adjacent to the VC/VS, SGC, or OFC. In some embodiments, an electrode array, arranged on a planar support substrate, may be used for electrically stimulating the VC/VS, SGC, or OFC region, or other region of the brain as specified herein. The surface area of the electrode array may be determined by the desired area of contact between the electrode array and the brain. An electrode for implanting on a brain surface, such as, a surface electrode or a surface electrode array may be obtained from a commercial supplier. A commercially obtained electrode/electrode array may be modified to achieve a desired contact area. In some cases, the non-brain penetrating electrode (also referred to as surface electrode) that may be used in the methods disclosed herein may be an electrocorticography (ECoG) electrode or an electroencephalography (EEG) electrode.

In certain cases, placing the electrode at a target area or site (e.g., a stimulation electrode) may involve positioning a brain penetrating electrode (also referred to as depth electrode) in the specified region(s) of the brain. For example, a stimulation electrode may be placed in the VC/VS, SGC, or OFC region, or another region. In some embodiments, the stimulation electrode may additionally contact area(s) adjacent to the VC/VS, SGC, or OFC. In some embodiments, an electrode array may be used for electrically stimulating the VC/VS, SGC, or OFC region, or another region of the brain as specified herein.

The depth to which a stimulation electrode is inserted into the brain may be determined by the desired level of contact between the electrode array and the brain. A brain-penetrating electrode array may be obtained from a commercial supplier. A commercially obtained electrode array may be modified to achieve a desired depth of insertion into the brain tissue.

In certain embodiments of the present methods, electrical stimulation is applied only to the VC/VS region in the brain. In other embodiments, electrical stimulation is applied at least to the VC/VS region in the brain and one or more additional regions in the brain.

In certain embodiments, one or more detection electrodes are used to record electroencephalographic signals for neural activity associated with a symptom of a neuropsychiatric disorder in one or more brain regions. A detection electrode may be placed, for example, in the right amygdala, left amygdala, right orbitofrontal cortex, left subgenual cingulate, or right hippocampus regions of the brain, or in other regions of the brain suitable for detection. The site chosen for detection may differ for different subjects and will depend on the mapping of the brain of an individual subject to identify the optimal location for positioning an electrode for detecting electroencephalographic signals from neural activity associated with a symptom of the neuropsychiatric disorder, as discussed further below.

In certain cases, placing the detection electrode may involve positioning the electrode on the surface of the specified region(s) of the brain. For example, electrodes may be placed on the surface of the brain at the right amygdala, left amygdala, right orbitofrontal cortex, left subgenual cingulate, or right hippocampus regions, or any combination thereof. The electrode may contact at least a portion of the surface of the brain at the right amygdala, left amygdala, right orbitofrontal cortex, left subgenual cingulate, or right hippocampus regions. In some embodiments, the electrode may contact substantially the entire surface area at the right amygdala, left amygdala, right orbitofrontal cortex, left subgenual cingulate, or right hippocampus regions. In some embodiments, the electrode may additionally contact area(s) adjacent to the right amygdala, left amygdala, right orbitofrontal cortex, left subgenual cingulate, or right hippocampus regions. In some embodiments, an electrode array arranged on a planar support substrate may be used for detecting electroencephalographic signals for neural activity from one or more of the brain regions specified herein. The surface area of the electrode array may be determined by the desired area of contact between the electrode array and the brain. An electrode for implanting on a brain surface, such as, a surface electrode or a surface electrode array may be obtained from a commercial supplier. A commercially obtained electrode/electrode array may be modified to achieve a desired contact area. In some cases, the non-brain penetrating electrode (also referred to as a surface electrode) that may be used in the methods disclosed herein may be an electrocorticography (ECoG) electrode or an electroencephalography (EEG) electrode. In certain embodiments, a plurality of electrodes is positioned at one or more of the brain regions specified herein for detection of electroencephalographic signals by stereoelectroencephalography (sEEG).

In certain cases, placing the detection electrode at a target area or site (e.g., the right amygdala, left amygdala, right orbitofrontal cortex, left subgenual cingulate, or right hippocampus) may involve positioning a brain penetrating electrode (also referred to as depth electrode) in the specified region(s) of the brain. For example, a detection electrode may be placed in the right amygdala, left amygdala, right orbitofrontal cortex, left subgenual cingulate, or right hippocampus region. In some embodiments, the detection electrode may additionally contact area(s) adjacent to the right amygdala, left amygdala, right orbitofrontal cortex, left subgenual cingulate, or right hippocampus. In some embodiments, an electrode array may be used for detecting neural activity from the right amygdala, left amygdala, right orbitofrontal cortex, left subgenual cingulate, or right hippocampus as specified herein.

The depth to which a detection electrode is inserted into the brain may be determined by the desired level of contact between the electrode array and the brain. A brain-penetrating electrode array may be obtained from a commercial supplier. A commercially obtained electrode array may, be modified to achieve a desired depth of insertion into the brain tissue.

The precise number of stimulation or detection electrodes contained in an electrode array (e.g., for electrical stimulation or detection of neural activity) may vary. In certain aspects, an electrode array may include two or more electrodes, such as 3 or more, including 4 or more. e.g., about 3 to 6 electrodes, about 6 to 12 electrodes, about 12 to 18 electrodes, about 18 to 24 electrodes, about 24 to 30 electrodes, about 30 to 48 electrodes, about 48 to 72 electrodes, about 72 to 96 electrodes, or about 96 or more electrodes. The electrodes may be arranged into a regular repeating pattern (e.g., a grid, such as a grid with about 1 cm spacing between electrodes), or no pattern. An electrode that conforms to the target site for optimal delivery of electrical stimulation may be used. One such example, is a single multi contact electrode with eight contacts separated by 2½ mm. Each contract would have a span of approximately 2 mm. Another example is an electrode with two 1 cm contacts with a 2 mm intervening gap. Yet further, another example of an electrode that can be used in the present methods is a 2 or 3 branched electrode to cover the target site. Each one of these three pronged electrodes has four 1-2 mm contacts with a center to center separation of 2 of 2.5 mm and a span of 1.5 mm.

The size of each electrode may also vary depending upon such factors as the number of electrodes in the array, the location of the electrodes, the material, the age of the patient, and other factors. In certain aspects, an electrode array has a size (e.g., a diameter) of about 5 mm or less, such as about 4 mm or less, including 4 mm-0.25 mm, 3 mm-0.25 mm, 2 mm-0.25 mm, 1 mm-0.25 mm, or about 3 mm, about 2 mm, about 1 mm, about 0.5 mm, or about 0.25 mm.

In certain embodiments, the method further comprises mapping the brain of the subject to optimize positioning of an electrode for applying electrical stimulation. Positioning of a stimulation electrode is optimized to maximize clinical responses to electrical stimulation to relieve symptoms of a neuropsychiatric disorder such as depression and/or anxiety. In some embodiments, the ventral capsule/ventral striatum (VC/VS) region, subgenual cingulate (SGC) region, orbitofrontal cortex (OFC) region, or other regions of the brain are mapped to determine optimal positioning of stimulation electrodes.

Assessment of the effectiveness of electrical stimulation at a particular site may, be performed by neurological examinations and/or neuropsychological tests (e.g., Minnesota Multiphasic Personality Inventory, Beck Depression Inventory, Mini-Mental Status Examination (MMSE), Visual Analogue Scale for Depression (VAS-D). Hamilton Depression Rating Scale (HAM-D), Montgomery-Asberg Depression Rating Scale (MADRS), Wisconsin Card Sorting Test (WCST), Tower of London, Stroop task, Montgomery-Asberg Depression Rating Scale (MADRS), Yale-Brown Obsessive Compulsive score (Y-BOCS)), motor examination, visual analog scales of pain symptoms, and/or cranial nerve examination.

In certain cases, the symptom that may be ameliorated by electrical stimulation is anxiety. Anxiety of a subject may be assessed using any standardized assessment method. In certain cases, anxiety may be measured by self-reporting, such as, by a Beck Anxiety Inventory (BAL) score, Visual Analogue Scale for Anxiety (VAS-A), or Hamilton Anxiety Rating Scale (HAM-A). Additional measures of anxiety include State-straight anxiety inventory (STAI) which consists of State Anxiety Scale (S-Anxiety) and Trait Anxiety Scale (T-Anxiety) and hospital anxiety and depression scale-Anxiety (HADS-A). Amelioration of anxiety may include a reduction in anxiety level compared to the anxiety level prior to the electrical stimulation.

In certain cases, the symptom that may be ameliorated by electrical stimulation is depression. Depression of a subject may be assessed using any standardized assessment method. In certain cases, depression may be measured by self-reporting, such as, by Visual Analogue Scale for Depression (VAS-D), Hamilton Depression Rating Scale (HAM-D), Montgomery-Asberg Depression Rating Scale (MADRS), a Beck Depression Inventory (BDI) score, Center for Epidemiological Studies Depression Scale (CES-D), Geriatric Depression Scale (GDS), or Zung Self-Rating Depression Scale (Zung SDS). In certain cases, an interviewer-administered depression assessment may be used alone or in conjunction with a self-reporting tool. Interviewer-administered depression assessments include Cornell Scale for Depression in Dementia (CSDD) and RAND Corporation Self-Administered Depression Screener Amelioration of depression may include a reduction in depression level compared to the depression level prior to the electrical stimulation.

In certain embodiments, the method further comprises mapping the brain of the subject to optimize positioning of a detection electrode. Positioning of the detection electrode is optimized to detect brain activity features that distinguish symptom states of a neuropsychiatric disorder that are severe enough to need treatment from symptomless states (baseline) or minor symptom states that do not need treatment. For example, the levels of overall power, or power in specific frequency ranges (e.g., alpha, delta, beta, gamma, and/or high gamma) may be correlated with symptom severity and need for treatment with electrical stimulation. In some embodiments, the level of gamma frequency power (such as 30 Hz to 200 Hz) is correlated with symptom severity to determine if a patient is in need of treatment with electrical stimulation. Thus, detection electrodes may be positioned to optimize detection of brain activity in specific frequency ranges that correlate with symptom severity of a neuropsychiatric disorder. Alternatively or additionally, coherence within certain spectral frequency bands or other features of network connectivity may be correlated with symptom severity and need for treatment with electrical stimulation.

Detection of brain activity may be performed by any method known in the art. For example, functional brain imaging of neural activity may be carried out by electrical methods such as electroencephalography (EEG), stereoelectroencephalography (sEEG), magnetoencephalography (MEG), single photon emission computed tomography (SPECT), as well as metabolic and blood flow studies such as functional magnetic resonance imaging (fMRI), and positron emission tomography (PET), In some embodiments, the right amygdala, left amygdala, right orbitofrontal cortex, left subgenual cingulate, or right hippocampus, or other regions are mapped to determine optimal positioning for detection electrodes. One or more of these regions may be implanted with detection electrodes to measure electrical signals from neural activity associated with a symptom of a neuropsychiatric disorder.

As set forth here, the method involves applying electrical stimulation in a manner effective to treat a neuropsychiatric disorder and/or a symptom of a neuropsychiatric disorder in a subject when neural activity associated with a symptom of the neuropsychiatric disorder is detected. Closed-loop therapy can be performed with a neurostimulator that records electroencephalographic signals from neural activity associated with a symptom of the neuropsychiatric disorder and delivers electrical stimulation when the level of an electroencephalographic signal that is detected exceeds a set threshold level indicating a level of symptom severity in need of treatment. The parameters for applying the electrical stimulation may be determined empirically during treatment or may be pre-defined, such as, from a clinical study. The parameters of the electrical stimulation may include one or more of frequency, pulse width/duration, duty cycle, intensity/amplitude, pulse pattern, program duration, program frequency, and the like.

Frequency refers to the pulses produced per second during stimulation and is stated in units of Hertz (Hz, e.g., 60 Hz=60 pulses per second). The frequencies of electrical stimulation used in the present methods may vary widely depending on the numerous factors and may be determined empirically during treatment of the subject or may be pre-defined. In certain embodiments, the method may involve applying an electrical stimulation at a frequency of 10 Hz-500 Hz, such as, 10 Hz-300 Hz, 10 Hz-200 Hz, 10 Hz-150 Hz, 10 Hz-125 Hz, 10 Hz-100 Hz, 15 Hz-200 Hz, 15 Hz-300 Hz, 20 Hz-200 Hz, 25 Hz-400 Hz, 25 Hz-300 Hz, 25 Hz-200 Hz, 25 Hz-150 Hz, 25 Hz-100 Hz, 50 Hz-500 Hz, 50 Hz-400 Hz, 50 Hz-300 Hz, 50 Hz-200 Hz, 50 Hz-150 Hz, 50 Hz-100 Hz, 75 Hz-300 Hz, 75 Hz-200 Hz, 75 Hz-150 Hz, 75 Hz-125 Hz, 75 Hz-120 Hz, 75 Hz-115 Hz, 75 Hz-110 Hz, or 75 Hz-100 Hz. The amplitude of current may be 0.1 mA-30 mA, such as, 0.1 mA-25 mA, such as, 0.1 mA-20 mA, 0.1 mA-15 mA, 0.1 mA-10 mA, 1 mA-20 mA, 1 mA-10 mA, 2 mA-30 mA, 2 mA-15 mA, or 2 mA-10 mA.

The electrical stimulation may be applied in pulses such as a uniphasic or a biphasic pulse. The time span of a single pulse is referred to as the pulse width or pulse duration. The pulse width used in the present methods may vary widely depending on numerous factors (e.g., severity of the disease, status of the patient, and the like) and may be determined empirically or may be pre-defined. In certain embodiments, the method may involve applying an electrical stimulation at a pulse width of about 10 μsec-990 sec, for example, 30 μsec-990 μsec, 50 μsec-990 μsec, 75 μsec-990 μsec, 100 μsec-990 μsec, 200 μsec-990 μsec, 300 μsec-990 μsec, 500 μsec-990 μsec, 500 μsec-900 μsec, 30 μsec-900 μsec, 50 μsec-900 μsec, 75 μsec-900 μsec, 100 μsec-900 μsec, 200 μsec-900 μsec, 300 μsec-900 μsec, 500 μsec-900 μsec, 30 μsec-500 μsec, 50 μsec-450 μsec, 75 μsec-300 μsec, 100 μsec-200 μsec, or 100 μsec-550 μsec. In some embodiments, the electrical stimulation is applied at a pulse width of about 100 μsec to about 140 μsec, including any pulse width within this range such as 100, 102, 104, 106, 108, 110, 112, 114, 116, 118, 120, 122, 124, 126, 128, 130, 132, 134, 136, 138, or 140 μsec.

The electrical stimulation may be applied for a stimulation period of 0.1 sec-1 month, with periods of rest (i.e., no electrical stimulation) possible in between. In certain cases, the period of electrical stimulation may be 0.1 sec-1 week, 1 sec-1 day, 10 sec-12 hours, 1 min-6 hours, 10 min-1 hour, and so forth. In certain cases, the period of electrical stimulation may be 1 sec-1 min, 1 sec-30 sec, 1 sec-15 sec, 1 sec-10 sec, 1 sec-6 sec, 1 sec-3 sec, 1 sec-2 sec, or 6 sec-10 sec. The period of rest in between each stimulation period may be 60 sec or less, 30 sec or less, 20 sec or less, or 10 sec.

The electrical stimulation may be applied with an amplitude of current of 0.1 mA-30 mA, such as, 0.1 mA-25 mA, such as, 0.1 mA-20 mA, 0.1 mA-15 mA, 0.1 mA-10 mA, 0.1 mA-2 mA, 0.1 mA-1 mA, 1 mA-20 mA, 1 mA-10 mA, 2 mA-30 mA, 2 mA-15 mA, 2 mA-10 mA, or 1 mA-2 mA. In some embodiments, the amplitude of current is 0.1 mA-1, 5 mA, or any amplitude of current in this range such as 0.1, 0.2, 0.3, 0.4, 0.5, 0.6, 0.7, 0.8, 0.9, 1.0, 1.1, 1.2, 1.3, 1.4, or 1.5 mA.

The electrical stimulation having the parameters as set forth above may be applied over a program duration of around 1 day or less, such as, 18 hours, 6 hours, 3 hours, 1 hour, 45 minutes, 30 minutes, 20 minutes, 10 minutes, or 5 minutes, or less, e.g., 1 minute-5 minutes, 2 minutes-10 minutes, 2 minutes-20 minutes, 2 minutes-30 minutes, 5 minutes-10 minutes, 5 minutes-30 minutes, or 5 minutes-15 minutes which period would include the application of pulses and the intervening rest period. The program may be repeated at a desired program frequency till treatment of the subject is achieved. As such, a treatment regimen may include a program for electrical stimulation at a desired program frequency and program duration. In some embodiments, the treatment regimen is controlled by a control unit in communication with a pulse generator connected to the one or more stimulation electrodes in a closed-loop treatment regimen.

In some embodiments, a cap on the maximum number of electrical stimulations per day can be set. For example, the maximum number of electrical stimulations per day may range from 50 therapies per day to 500 therapies per day, including any number of therapies per day in this range such as 50, 75, 100, 125, 150, 175, 200, 225, 250, 275, 300, 325, 350, 375, 400, 425, 450, 475, or 500 therapies per day. Alternatively or additionally a cap can be set on the total amount of time of electrical stimulation per day. For example, the total amount of time of electrical stimulation per day may range from 10 minutes to 100 minutes of total stimulation per day, including any amount of time within this range such as 10, 20, 30, 40, 50, 60, 70, 80, 90, or 100 minutes of total stimulation time per day. Such caps on maximum number of electrical stimulations and/or total amount of time of electrical stimulation per day can be employed so as not to disturb the sleep of a patient from evening therapy. In addition, a rest period without electrical stimulation when a patient wishes to sleep may also be employed to avoid interfering with sleep.

As noted above, the treatment may ameliorate one or more symptoms of the neuropsychiatric disorder suffered by the subject. Such symptoms include anxiety, depression, frequency of episodes of compulsive behavior, frequency of self-starvation episodes, mania, panic attacks, social anxiety, or distress related to chronic pain. Assessment of effectiveness of the treatment may be performed by neurological examinations and/or neuropsychological tests (e.g., Minnesota Multiphasic Personality Inventory. Beck Depression Inventory, Mini-Mental Status Examination (MMSE). Visual Analogue Scale for Depression (VAS-D). Hamilton Depression Rating Scale (HAM-D), Montgomery-Asberg Depression Rating Scale (MADRS), Wisconsin Card Sorting Test (WCST), Tower of London, Stroop task, Montgomery-Asberg Depression Rating Scale (MADRS), Yale-Brown Obsessive Compulsive score (Y-BOCS)), motor examination, visual analog scales of pain symptoms, and/or cranial nerve examination.

In certain cases, the symptom that may be ameliorated by the disclosed method may be anxiety. Anxiety of a subject may be assessed using any standardized assessment method. In certain cases, anxiety may be measured by self-reporting, such as, by a Beck Anxiety Inventory (BAI) score, Visual Analogue Scale for Anxiety (VAS-A), or Hamilton Anxiety Rating Scale (HAM-A). Additional measures of anxiety include State-straight anxiety inventory (STAI) which consists of State Anxiety Scale (S-Anxiety) and Trait Anxiety Scale (T-Anxiety) and hospital anxiety and depression scale-Anxiety (HADS-A). Amelioration of anxiety may include a reduction in anxiety level compared to the anxiety level prior to the treatment. A 5% or higher reduction in anxiety level (measured by BAI or HAM-A, for example), may indicate that the treatment was effective.

In certain cases, the symptom that may be ameliorated by the disclosed method may be depression. Depression of a subject may be assessed using any standardized assessment method. In certain cases, depression may be measured by self-reporting, such as, by Visual Analogue Scale for Depression (VAS-D), Hamilton Depression Rating Scale (HAM-D), Montgomery-Asberg Depression Rating Scale (MADRS), a Beck Depression Inventory (BDI) score, Center for Epidemiological Studies Depression Scale (CES-D). Geriatric Depression Scale (GDS), or Zung Self-Rating Depression Scale (Zung SDS). In certain cases, an interviewer-administered depression assessment may be used alone or in conjunction with a self-reporting tool. Interviewer-administered depression assessments include Cornell Scale for Depression in Dementia (CSDD) and RAND Corporation Self-Administered Depression Screener Amelioration of depression may include a reduction in depression level compared to the depression level prior to the treatment. A 5% or higher reduction in depression level (measured by BDI score, for example), may indicate that the treatment was effective.

In certain cases, effectiveness of treatment may be assessed by detecting activity (e.g., electrical signals) from a secondary region of the brain, which may be within the amygdala, or another area. For example, the secondary region(s) may be one or more of the amygdala, orbitofrontal cortex, hippocampus, septum, cingulate gyrus, cingulate cortex, subgenual cingulate, hypothalamus, epithalamus, anterior thalamus, mammillary bodies, and fornix. In certain embodiments, the secondary region may be a right amygdala region, left amygdala region, a right orbitofrontal cortex region, a left subgenual cingulate region, or a right hippocampus region of the brain of the subject, or any combination thereof. Detection of brain activity at a secondary region of the brain may be performed by functional brain imaging. Functional brain imaging may be carried out by electrical methods such as electroencephalography (EEG), magnetoencephalography (MEG), single photon emission computed tomography (SPECT), as well as metabolic and blood flow studies such as functional magnetic resonance imaging (fMRI), and positron emission tomography (PET). In some embodiments, electrical methods for assessing effectiveness of treatment may involve use of a detection electrode as described herein or placement of an additional electrode for measuring electrical signals at a secondary region of the brain. One or more regions of the brain may be implanted with an electrode and electrical signals measured for assessment of effectiveness of the treatment. Any suitable electrodes may be used for measurements and may include one or more surface electrodes (non-brain penetrating electrode(s)) or one or more depth electrodes (brain penetrating electrode(s)) as described herein.

Assessment of effectiveness of treatment and assessment of amelioration of a symptom of the neuropsychiatric disorder may be performed at any suitable time point after commencement of the treatment procedure, for example, during closed-loop therapy or after a treatment regimen is complete. Embodiments of the subject methods include assessing effectiveness of treatment or amelioration of a symptom of the neuropsychiatric disorder within seconds, minutes, hours, or days after the initial treatment regimen has been completed. In some instances, assessment may be performed at multiple time points. In some cases, more than one type of assessment may be performed at the different time points. In some embodiments, a subject's brain activity (e.g., at one or more secondary regions) may be measured prior to the application of electrical stimulation, and assessing may include comparing the subject's brain activity at one or more secondary regions after the treatment to that before the treatment and a change in the post-treatment brain activity may indicate successful treatment.

In some embodiments, the subject is monitored for a period of time during closed-loop therapy using a detection electrode, wherein increasing numbers of the electroencephalographic signals exceeding the threshold level detected by the detection electrode within a later recording period compared to an earlier recording period indicate increasing severity of a symptom of the neuropsychiatric disorder; and decreasing numbers of electroencephalographic signals exceeding the threshold level detected by the detection electrode within a later recording period compared to an earlier recording period indicate decreasing severity of the symptom of the neuropsychiatric disorder.

The methods and systems provided herein may be used to ameliorate one or more symptoms of a neuropsychiatric disorder in a patient. A person skilled in the art will appreciate that amelioration of a symptom may provide relief to a patient suffering from two separate neuropsychiatric conditions which share the symptom.

Upon completion of a treatment regimen, the patient may be assessed for effectiveness of the treatment and the treatment regimen may be repeated, if needed. In certain cases, the treatment regimen may be altered before repeating. For example, one or more of the frequency, pulse width, current amplitude, period of electrical stimulation, program duration, program frequency, and/or placement of stimulation or detection electrodes may be altered before starting a second treatment regimen.

In certain cases, the treatment regimen may be tailored to the desired outcome. For example, for a patient suffering from an acute form of a neuropsychiatric disorder(s), the treatment regimen may be chosen to provide an acute alleviation of one or more symptoms of the disorder(s). In contrast, the treatment regimen for a patient suffering chronically from a neuropsychiatric disorder(s) may be tailored for a chronic relief of one or more symptoms of the disorder(s). A treatment regimen for chronic relief of one or more symptoms of the disorder(s) may also delay reappearance of symptoms of the chronic relief of one or more symptoms of the disorder(s) and may even prevent the symptoms from appearing. In certain cases, a treatment regimen that is a combination of treatment regimens for acute and chronic alleviation of one or more symptoms of the disorder(s) may be employed.

Application of the method may include a prior step of selecting a patient for treatment based on need as determined by clinical assessment, which may include cognitive assessment, anatomical assessment, behavioral assessment and/or neurophysiological assessment. In certain cases, a subject may be selected for treatment if the subject is at risk of suffering from a neuropsychiatric disorder. A patient who has a family history of neuropsychiatric disorders or has previously suffered from neuropsychiatric disorders may be at risk. Such a patient may also be implanted with detection and stimulation electrodes.

In certain aspects, the methods and systems of the present disclosure may include measurement of brain activity, for example, electrical activity in the amygdala, where the level of gamma-frequency power may be measured. In certain cases, electrical activity from a plurality of locations in the amygdala may be measured and averaged. In some embodiments, electrical activity in the gamma frequency range (such as 30 Hz to 200 Hz) may be measured from the right amygdala, left amygdala, right orbitofrontal cortex, left subgenual cingulate, or right hippocampus, or any combination thereof. In some cases, electrical activity in one or more locations in the brain may be measured during a period extending from prior to stimulation to the period during which stimulation to the brain is applied, or to a period after stimulation to the brain has been applied, and monitored for a decrease in the power of gamma-frequency (such as 30 Hz to 200 Hz) activity and/or increase in cortical excitability. In some cases, when decreased power of gamma-frequency (such as 30 Hz to 200 Hz) activity and/or increased cortical excitability is within a normal range (e.g., a range associated with a substantial lack of the disorder), the methods and systems do not apply a further stimulation to the brain. Alternatively, when decreased power of gamma-frequency (such as 30 Hz to 200 Hz) activity and/or increased cortical excitability is not within anormal range (e.g., a range associated with a substantial lack of the disorder), the methods and systems may apply a further stimulation to the brain. In certain cases, the application of electrical stimulation to the brain may suppress gamma-frequency power across the right amygdala, left amygdala, right orbitofrontal cortex, left subgenual cingulate, or right hippocampus. The decrease may be as compared to the power prior to the application of stimulation. In certain cases, the application of electrical stimulation to the brain may alter other neural features from one more regions of the brain. The alterations may be compared to the state of these features prior to the application of stimulation.

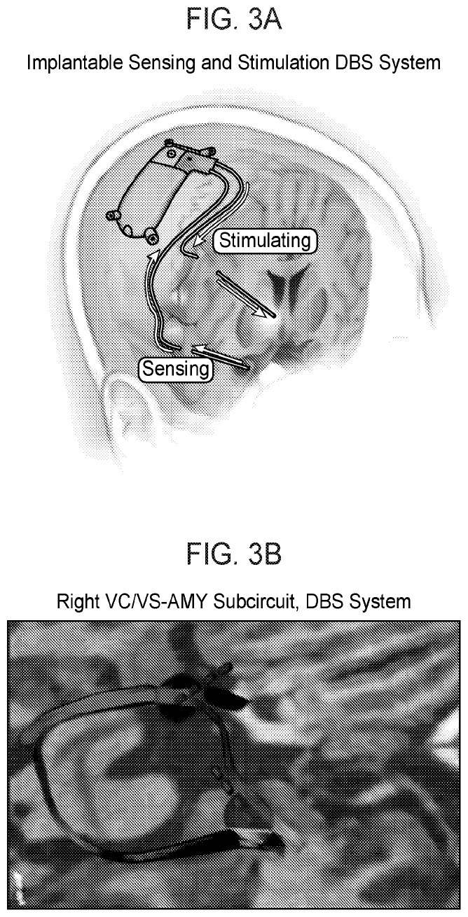

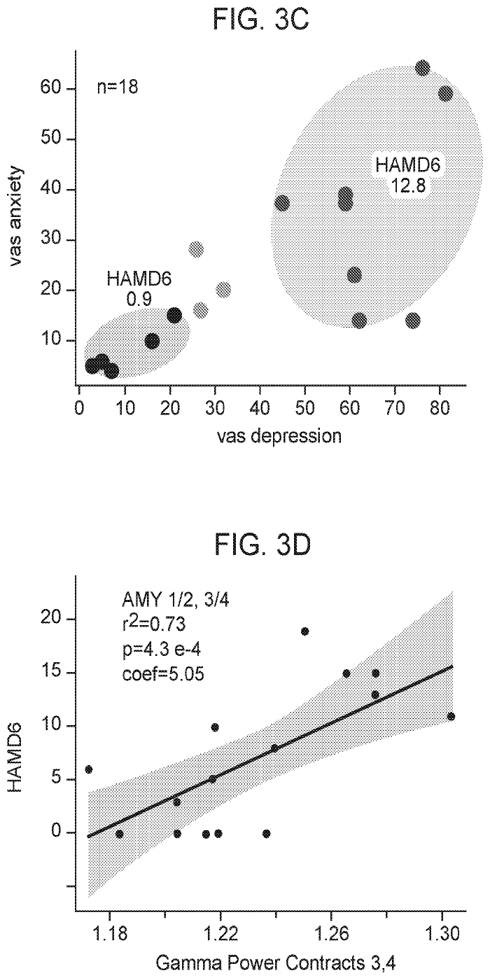

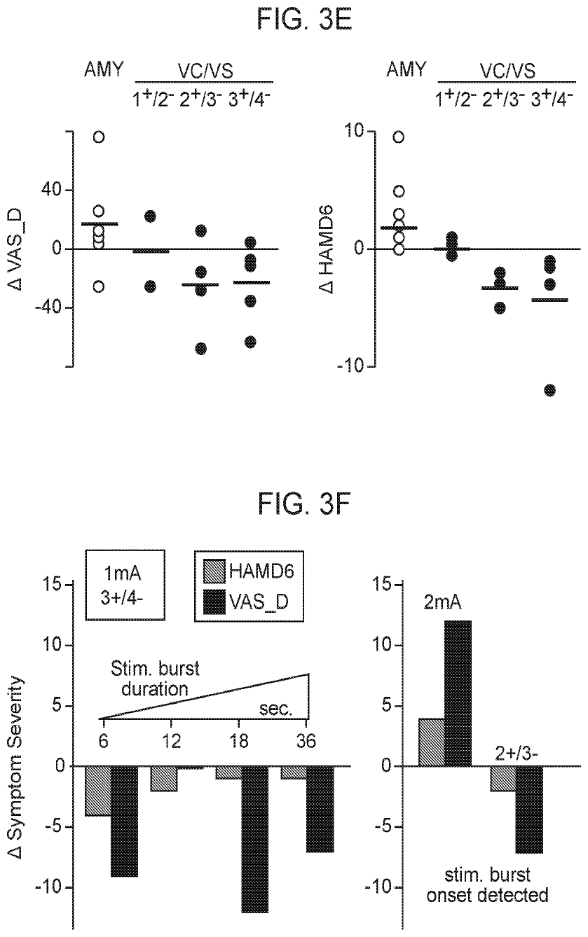

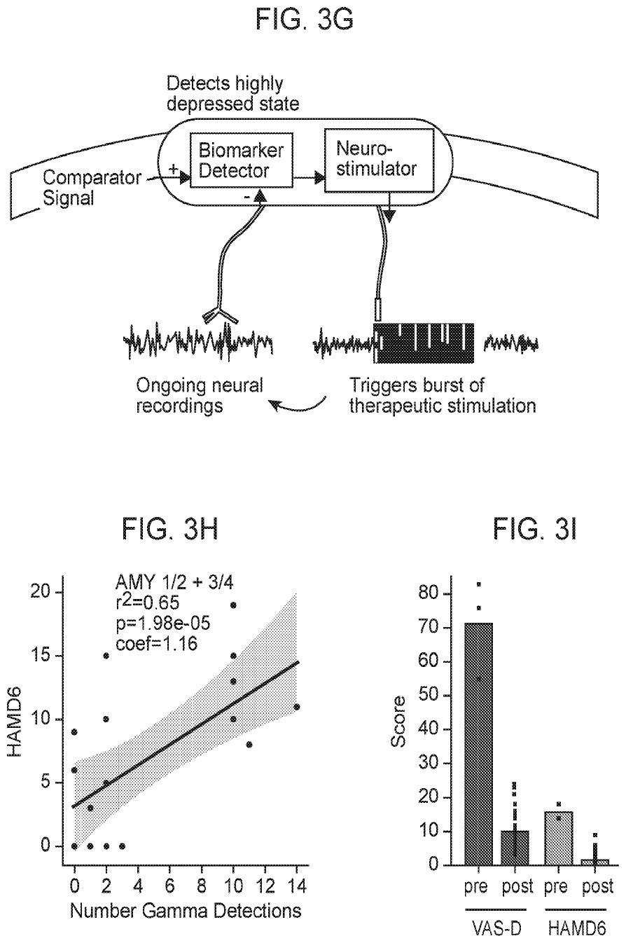

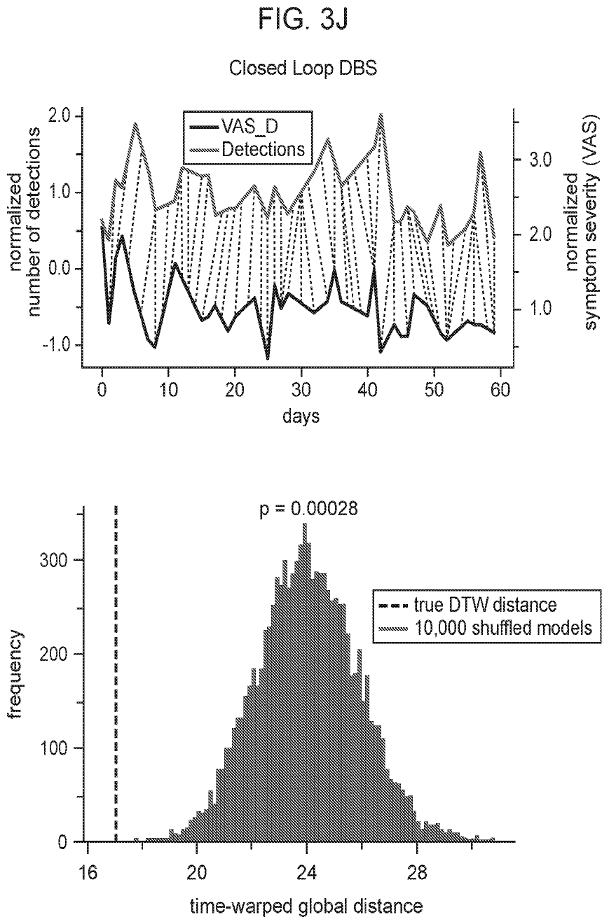

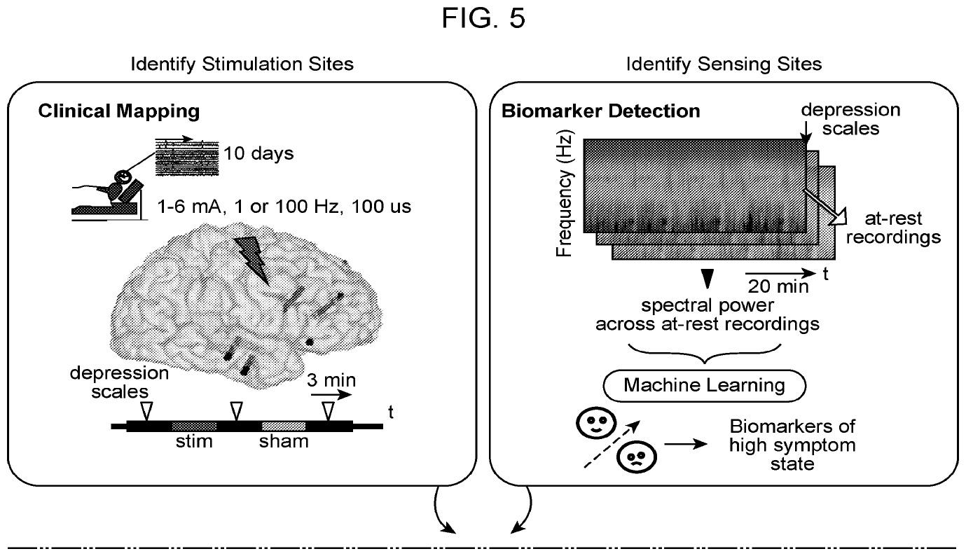

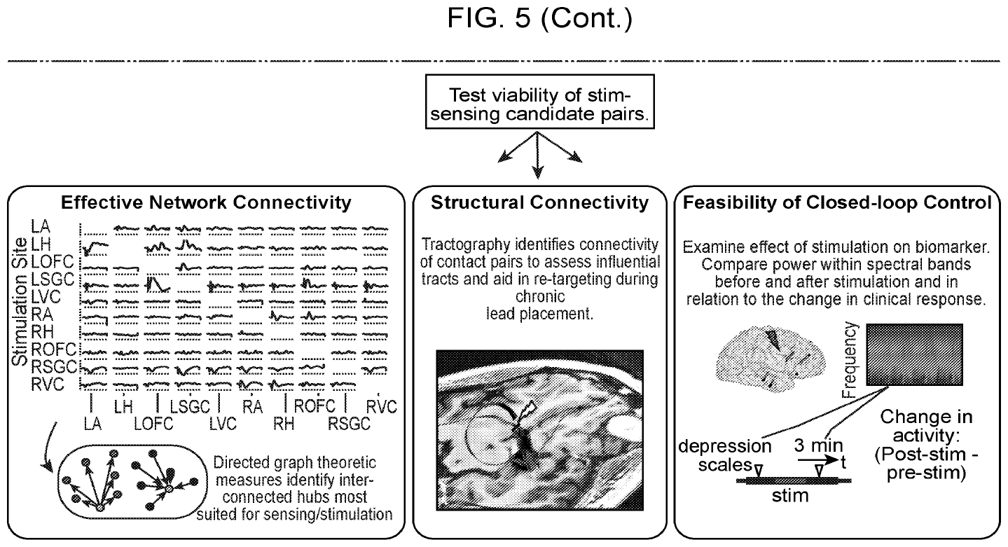

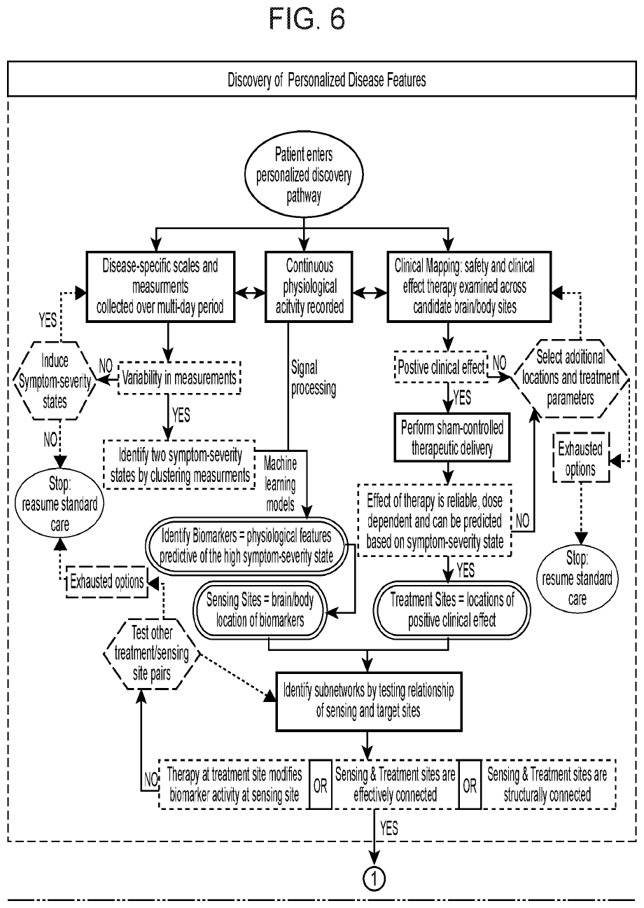

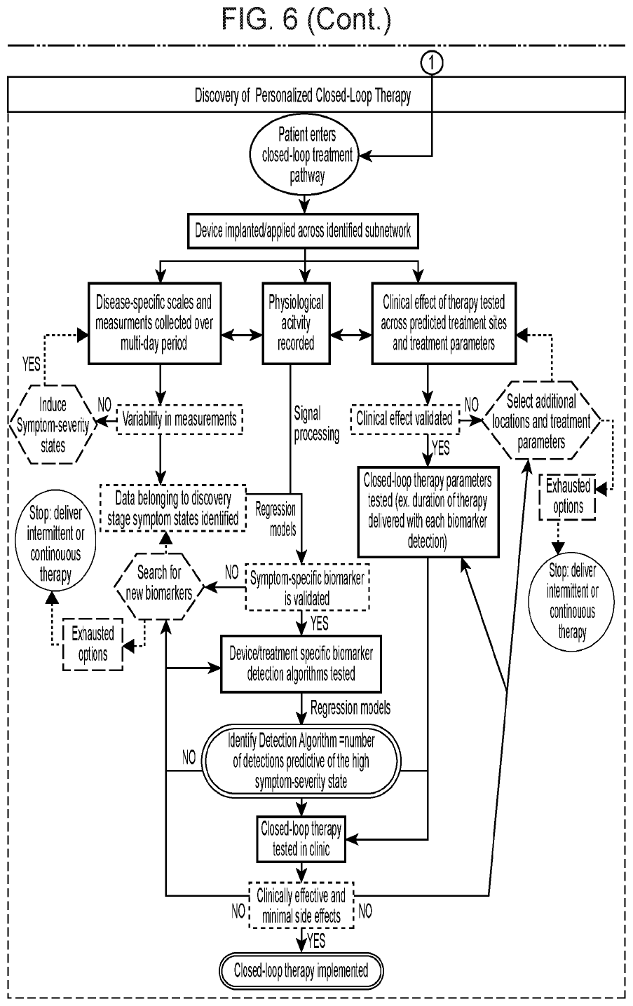

A closed-loop method allows determination of parameters of electrical stimulation based upon real-time feedback signals from the brain of the subject. Closed-loop methods and systems allow for automation of treatment of the subject including real-time need-based modulation of the treatment regimen. Exemplary closed-loop methods and associated systems for treatment of a neuropsychiatric disorder are further discussed in the Examples section and are depicted in FIG. 3 and FIG. 5. Closed-loop methods and systems for automated delivery of electrical stimulation are further described below.

### Closed-Loop Method for Automated Delivery of Electrical Stimulation

In certain embodiments, a control algorithm is used to automate the delivery of electrical stimulation in response to detection of neural activity associated with a symptom of a neuropsychiatric disorder. According to certain embodiments, the method may include receiving an electrical signal from a region of the brain of the subject via a detection electrode; applying electrical signal metrics to a control algorithm that is tuned to a clinically relevant target (e.g., a range of signal indicative of effective treatment and/or a range of signal indicative of severity of symptoms and the need for treatment); automatically delivering electrical stimulation to the brain via the stimulation electrode in a manner effective to treat the neuropsychiatric disorder or ameliorate a symptom of the neuropsychiatric disorder if the electrical signal metrics indicate that the patient is in need of treatment. For example, electrical activity in the gamma frequency range (such as 30 Hz to 200 Hz) from the right amygdala, left amygdala, right orbitofrontal cortex, left subgenual cingulate, or right hippocampus may be measured with a detection electrode, wherein the control algorithm receives the electrical activity data from the detection electrode and automates delivery of electrical stimulation via a stimulation electrode to the VC/VS region when the level of gamma frequency power (such as 30 Hz to 200 Hz) is in a certain range indicating that a patient has a symptom of a neuropsychiatric disorder severe enough that the patient is in need of treatment. In some embodiments, one or more programmed stimulation parameters are modulated according to the algorithm's control law based on the recorded electrical activity data; and modulated electrical stimulation is delivered to the VC/VS region via the stimulation electrode in a manner effective to ameliorate the symptom of the neuropsychiatric disorder.

As described in the foregoing sections, effectiveness of treatment or amelioration of a symptom of the neuropsychiatric disorder may be assessed by detecting electrical activity from the second region(s) of the brain using a detection electrode. In an open-loop system, stimulation is delivered in a pre-programmed way or manually by a user but is not automatically controlled by real-time neural feedback from the patient's brain. The electrical activity may be analyzed by a computing means which may output recommendations based on comparing the electrical activity to a predetermined range. A user may then carry out the recommendations, such as, changing a parameter of the electrical stimulation program prior to starting another treatment regimen. In a closed-loop system, by contrast, a computing means can automatically update stimulation parameters based upon analysis of the recorded electrical signal and/or automatically deliver stimulation according to the electrical stimulation program. In some embodiments, either an open-loop or a closed-loop system may be integrated with a mechanism for user intervention, for example by allowing user-override of open-loop or closed-loop stimulation programs to enact or prevent stimulation that would ordinarily occur, or to manually change parameters of such stimulation.

In some embodiments, the computing means for directing closed-loop stimulation may be a combination of hardware/software which may be connected wirelessly or by wire to the measurement electrodes. The computing means may communicate with a control unit (also referred to as a control module) that controls a pulse generator connected to the stimulation electrodes. In certain embodiments, the computing means may be connected to a recorder (e.g., a neurophysiological recorder) that records brain activity measured by the detection electrodes. The computing means may include a control algorithm that determines modification of stimulation parameters based on real-time outputs of the neurophysiological recorder. The algorithm may operate by simple on/off control of stimulation at set parameters, modifying only the on/off parameter with each evaluation cycle, or may determine sophisticated modification of a range of stimulation parameters with each cycle. In some cases, the algorithm may be based on information related to the neuropsychiatric disorder, such as, a range of electrical activity that is indicative of the presence of the neuropsychiatric disorder or severity of symptoms. The algorithm may also include additional information such as a brain activity profile of anormal subject (not suffering from a neuropsychiatric disorder). Regardless of the particular control algorithm structure, the computing means may be tuned to a clinically relevant target (e.g., a range of signal indicative of effective treatment and/or a range of signal indicative of severity of symptoms and the need for treatment) that directs modulation of one or more programmed stimulation parameters according to the algorithm's control law, applying the modulated electrical stimulation (e.g., to the VC/VS, SGC, or OFC region, or other region) via the stimulation electrode.

In some cases, the computing means, via a control algorithm, may determine whether the received electrical signals are within or outside a predetermined range of neural signals indicative of the presence of the neuropsychiatric disorder. When the received electrical signals are outside this predetermined range, then the computing means determines that the neuropsychiatric disorder has been treated and/or a symptom of the neuropsychiatric disorder (e.g., depression, anxiety, compulsive/repetitive, self-starvation behavior, and/or chronic-pain-related distress) has been ameliorated. The computing means may then communicate with the control unit to direct stimulation shut-off by the pulse generator. When the received electrical signals are within the predetermined range of neural signals indicative of the presence of the neuropsychiatric disorder, then the computing means determines that the neuropsychiatric disorder is in need of treatment and/or a symptom of the neuropsychiatric disorder (e.g., depression, anxiety, compulsive/repetitive, self-starvation behavior, and/or chronic-pain-related distress) is severe enough to require treatment. The control algorithm within the computing means may then determine whether the initial step of applying electrical stimulation should be repeated and/or whether a parameter of the electrical stimulation should be modified prior to the step of applying electrical stimulation. The computing means, via the control unit, may then communicate with the control unit to provide the appropriate instructions to the pulse generator.

In some embodiments, the computing means may determine whether the received electrical signals are within or outside a second predetermined range, where the second predetermined range is indicative of treatment of the neuropsychiatric disorder and/or amelioration of a symptom of the neuropsychiatric disorder. When the received electrical signals are within the second predetermined range, then the computing means determines that the neuropsychiatric disorder has been treated and/or a symptom of the neuropsychiatric disorder (e.g., depression, anxiety, compulsive/repetitive, self-starvation behavior, and/or chronic-pain-related distress) has been ameliorated. The computing means may then communicate with the control unit to direct stimulation switch-off by the pulse generator. When the received electrical signals are outside the second predetermined range, then the computing means determines that the neuropsychiatric disorder has not been treated and/or a symptom of the neuropsychiatric disorder (e.g., depression, anxiety, compulsive/repetitive, self-starvation behavior, and/or chronic-pain-related distress) has not been ameliorated. The control algorithm within the computing means may then determine whether the initial step of applying electrical stimulation should be repeated and/or whether a parameter of the electrical stimulation modified prior to the step of applying electrical stimulation. The processor may then communicate with the control unit to provide the appropriate instructions to the pulse generator.

Thus, in certain aspects, the subject methods operate as a closed-loop control system which may automatically adjust one or more parameters in response to electrical activity from a region of the brain of a subject and/or automatically deliver stimulation according to the electrical stimulation program. In some embodiments, the closed-loop control system automatically delivers stimulation according to set parameters when the received electrical signals are within a predetermined range indicative of the need for treatment. Exemplary closed-loop methods and associated systems are described in the Examples section of the application and are illustrated in FIG. 3 and FIG. 5.

In some aspects, the closed loop system may be used to sense a subject's need for treatment using the methods disclosed herein. For example, the closed loop system may be programmed to monitor brain activity from one or more regions of the brain and compare the brain activity to a range indicative of the neuropsychiatric disease or a symptom of the neuropsychiatric disease. Upon detection of electrical activity indicative of the neuropsychiatric disease or a symptom of the neuropsychiatric disease, the closed loop system may automatically commence a treatment protocol of applying electrical stimulation.

In additional aspects, the closed loop system may be used as a system for monitoring brain activity and correlating the brain activity to the subject's mental state. For example, since the closed loop system is configured for recording electrical signals from a subject's brain, the subject's mood and/or behavior may be monitored in real-time and correlated to the measured electrical signals to provide a biomarker that is related to the subject's mental state. For example, electrical activity measured when a subject is anxious or depressed can be used to develop a biomarker, e.g., as range of electrical activity indicative of anxiety or depression, and so on. As such, closed loop systems are useful for prognosis as well as diagnosis of neuropsychiatric disorders.

It is understood that electrical signals that are indicative of a mental state of a subject may be recorded from a subject's brain and may be used in aspects outside of a closed loop system. For example, electrical signals indicative of a mental state of a subject may be recorded from the right amygdala, left amygdala, right orbitofrontal cortex, left subgenual cingulate, or right hippocampus, or other region using electrodes or another device operably coupled to the patient's brain which electrodes or device may or may not be part of a closed loop system. The patient may be treated as disclosed herein (e.g., by applying electrical stimulation to the VC/VS, SGC, or OFC region, or other region), and electrical signals recorded from the same region in real time as the treatment is administered or after the treatment is administered. The electric signals recorded after the administration of electrical stimulation is commenced may then be compared to the electric signals recorded prior to the treatment to determine features in the recorded electric signals that change post-treatment. These features provide a feedback signal to indicate whether the treatment is having an effect on the patient's mental state. These features can also serve as feedback signals to a closed loop system. These features may include the overall power, or power in specific frequency ranges (e.g. alpha, delta, beta, gamma, and/or high gamma). In some cases, these features may be patient specific or specific to a particular mental state or both. For example, some of the features may be features found in a plurality of patients having a particular mental state (e.g., anxiety or depression); some of the features may be features in a particular patient which may not be found in a significant number of other patients having the same mental state. In some embodiments, a combination of patient-specific features and mental state specific features may be monitored to assess efficacy of treatment.

In a particular aspect, the closed loop system and methods provided herein may involve a recording of electrical signals from one or more regions (e.g., right amygdala, left amygdala, right orbitofrontal cortex, left subgenual cingulate, or right hippocampus) of a patient's brain, where the patient has a neuropsychiatric disorder. The patient may then be treated by application of electrical stimulation to the VC/VS, SGC, or OFC region, or other region of the brain, and electrical signals may be recorded from the same regions of the brain (e.g., right amygdala, left amygdala, right orbitofrontal cortex, left subgenual cingulate, or right hippocampus) and compared to the pre-treatment recording. Features in the recorded signals that changed after the treatment would correspond to biomarkers that indicate whether the treatment is having an effect. The change in recorded signals can also optionally be correlated to the mental state reported by the patient after the treatment. The change can be used for modulating the treatment in a closed loop system. For example, when the change in the recorded signal correlates with an improvement in the patient's mood, those features would indicate to a computing means of a closed loop system that further treatment need not be performed.

In certain aspects, methods of the present disclosure that may be embodied in an open- or a closed-loop system may include measuring brain activity from a subject having or suspected or having a neuropsychiatric disorder, such as, anxiety and/or depression, where when the subject has a level of gamma range frequencies (e.g., 30 Hz to 200 Hz) that is higher than a normal range (e.g., range reflective of a normal brain, such as, brain of a person not suffering from the disorder) or higher than a threshold level (e.g., indicating a symptom severe enough that treatment is needed), the subject is treated by applying electrical stimulation to the VC/VS. SGC, or OFC region, or other region via an electrode positioned at or adjacent to the VC/VS. SGC, or OFC region, or other region in a manner effective to reduce the level of gamma range frequencies in the brain of the subject. The brain activity may be measured at one or more brain regions (e.g., the right amygdala, left amygdala, right orbitofrontal cortex, left subgenual cingulate, or right hippocampus, or any combination thereof) In some aspects, the system may monitor brain activity such as levels of gamma frequency activity, and if the level of gamma frequency activity is higher than a reference range (e.g., range reflective of a normal brain, such as, brain of a person not suffering from the disorder) and/or higher than a threshold level (e.g., indicating a symptom severe enough that treatment is needed), the closed loop system may apply electrical stimulation to the VC/VS, SGC, or OFC region, or other region via an electrode positioned at or adjacent to the VC/VS, SGC, or OFC region, or other region in a manner effective to reduce the level of gamma frequency activity (e.g., 30 Hz to 200 Hz) in the brain of the subject.

In some embodiments, one or more pattern recognition methods can be used in analyzing recorded brain electrical activity data to automate detection of brain activity features that distinguish symptom states of a neuropsychiatric disorder that are severe enough to need treatment from symptomless states (baseline) or minor symptom states that do not need treatment. The models and/or algorithms can be provided in machine readable format and may be used to correlate the levels of overall power, or power in specific frequency ranges (e.g., alpha, delta, beta, gamma, and/or high gamma) with symptom severity and need for treatment with electrical stimulation. In some embodiments, the level of gamma frequency power (such as 30 Hz to 200 Hz) is correlated with symptom severity to determine if a patient is in need of treatment with electrical stimulation. Alternatively or additionally, coherence within certain spectral frequency bands or other features of network connectivity may be correlated with symptom severity and need for treatment with electrical stimulation.

Analyzing the recorded brain electrical activity may comprise the use of an algorithm or classifier. In some embodiments, a machine learning algorithm is used to classify brain activity as indicating whether or not a symptom of a neuropsychiatric disorder is severe enough that electrical stimulation should be delivered to the brain of the patient. The machine learning algorithm may comprise a supervised learning algorithm. Examples of supervised learning algorithms may include Average One-Dependence Estimators (AODE), Artificial neural network (e.g., Backpropagation), Bayesian statistics (e.g., Naive Bayes classifier, Bayesian network, Bayesian knowledge base), Case-based reasoning, Decision trees, Inductive logic programming, Gaussian process regression, Group method of data handling (GMDH), Learning Automata, Learning Vector Quantization, Minimum message length (decision trees, decision graphs, etc.), Lazy learning, Instance-based learning Nearest Neighbor Algorithm, Analogical modeling, Probably approximately correct learning (PAC) learning, Ripple down rules, a knowledge acquisition methodology, Symbolic machine learning algorithms, Subsymbolic machine learning algorithms, Support vector machines, Random Forests, Ensembles of classifiers, Bootstrap aggregating (bagging), and Boosting. Supervised learning may comprise ordinal classification such as regression analysis and Information fuzzy networks (IFN). Alternatively, supervised learning methods may comprise statistical classification, such as AODE. Linear classifiers (e.g., Fisher's linear discriminant, Logistic regression, Naive Bayes classifier, Perceptron, and Support vector machine), quadratic classifiers, k-nearest neighbor, Boosting, Decision trees (e.g., C4.5, Random forests), Bayesian networks, and Hidden Markov models.

The machine learning algorithms may also comprise an unsupervised learning algorithm. Examples of unsupervised learning algorithms may include artificial neural network, Data clustering, Expectation-maximization algorithm, Self-organizing map, Radial basis function network, Vector Quantization, Generative topographic map, Information bottleneck method, and IBSEAD. Unsupervised learning may also comprise association rule learning algorithms such as Apriori algorithm, Eclat algorithm and FP-growth algorithm. Hierarchical clustering, such as Single-linkage clustering and Conceptual clustering, may also be used. Alternatively, unsupervised learning may comprise partitional clustering such as K-means algorithm and Fuzzy clustering.

In some instances, the machine learning algorithms comprise a reinforcement learning algorithm. Examples of reinforcement learning algorithms include, but are not limited to, temporal difference learning, Q-learning and Learning Automata. Alternatively, the machine learning algorithm may comprise Data Pre-processing.

### Administration of a Pharmacological Agent

Embodiments of the methods and systems provided in this disclosure may also include administration of an effective amount of at least one pharmacological agent. By “effective amount” is meant a dosage sufficient to prevent or treat a neuropsychiatric disorder in a subject as desired. The effective amount will vary somewhat from subject to subject, and may depend upon factors such as the age and physical condition of the subject, severity of the neuropsychiatric disorder being treated, the duration of the treatment, the nature of any concurrent treatment, the form of the agent, the pharmaceutically acceptable carrier used if any, the route and method of delivery, and analogous factors within the knowledge and expertise of those skilled in the art. Appropriate dosages may be determined in accordance with routine pharmacological procedures known to those skilled in the art, as described in greater detail below.

If a pharmacological approach is employed in the treatment of a neuropsychiatric disorder, the specific nature and dosing schedule of the agent will vary depending on the particular nature of the disorder to be treated. Representative pharmacological agents that may find use in certain embodiments of the subject invention include, but are not limited to, selective serotonin reuptake inhibitors (SSRIs) such as fluoxetine (Prozac) paroxetine (Paxil, Pexeva), sertraline (Zoloft), citalopram (Celexa) and escitalopram (Lexapro), serotonin-norepinephrine reuptake inhibitors (SNRIs) (e.g., duloxetine (Cymbalta), venlafaxine (Effexor XR), desvenlafaxine (Pristiq, Khedezla) and levomilnacipran (Fetzima), and the like.

In certain aspects, the administration of a pharmacological agent involves using a pharmacological delivery device such as, but not limited to, pumps (implantable or external devices), epidural injectors, syringes or other injection apparatus, catheter and/or reservoir operatively associated with a catheter, etc. For example, in certain embodiments a delivery device employed to deliver at least one pharmacological agent to a subject may be a pump, syringe, catheter or reservoir operably associated with a connecting device such as a catheter, tubing, or the like. Containers suitable for delivery of at least one pharmacological agent to a pharmacological agent administration device include instruments of containment that may be used to deliver, place, attach, and/or insert the at least one pharmacological agent into the delivery device for administration of the pharmacological agent to a subject and include, but are not limited to, vials, ampules, tubes, capsules, bottles, syringes and bags. Administration of a pharmacological agent may be performed by a user or by a closed loop system.

### Systems

The present disclosure also provides systems which find use, e.g., in practicing the subject methods. The system may be a closed-loop system configured for performing the methods provided herein. In some embodiments, the closed-loop system may include a stimulation electrode adapted for positioning at the VC/VS, SGC, or OFC region, or other region of the brain of the subject and a detection electrode adapted for positioning at a secondary area of the brain (e.g., right amygdala, left amygdala, right orbitofrontal cortex, left subgenual cingulate, or right hippocampus, or other region) and for recording an electrical signal from the secondary area before, during, or after an electrical stimulation is applied to the VC/VS, SGC, or OFC region, or other region. The system may also include a computing means and control unit programmed to instruct the stimulation electrode to apply an electrical stimulation to the VC/VS, SGC, or OFC region, or other region in a manner effective to treat the neuropsychiatric disorder in the subject and/or ameliorate a symptom of the neuropsychiatric disorder (e.g., depression, anxiety, compulsive behavior, and the like); receive the electrical signals from the secondary area of the brain of the subject via the detection electrode; apply electrical signal metrics to a control algorithm that is tuned to a clinically relevant target (e.g., a range of signal indicative of severity of symptoms and the need for treatment); and automatically delivering electrical stimulation to the brain via the control unit, pulse generator and stimulation electrode in a manner effective to treat the neuropsychiatric disorder or ameliorate a symptom of the neuropsychiatric disorder if the electrical signal metrics indicate that the patient is in need of treatment. For example, electrical activity in the gamma frequency range (such as 30 Hz to 200 Hz) from the right amygdala, left amygdala, right orbitofrontal cortex, left subgenual cingulate, or right hippocampus, or other region may be measured with a detection electrode using this system, wherein the control algorithm receives the electrical activity data from the detection electrode and automates delivery of electrical stimulation via the control unit, a pulse generator and the stimulation electrode to the VC/VS, SGC, or OFC region, or other region when the level of gamma frequency power (such as 30 Hz to 200 Hz) is in a certain range indicating that a patient has a symptom of a neuropsychiatric disorder severe enough that the patient is in need of treatment. In some embodiments, one or more programmed stimulation parameters are modulated according to the algorithm's control law based on the recorded electrical activity data, and modulated electrical stimulation is delivered to the VC/VS, SGC, or OFC region, or other region via the control unit, pulse generator and stimulation electrode in a manner effective to ameliorate the symptom of the neuropsychiatric disorder. The closed loop system may include an on-body pulse generator that is connected to the implanted stimulation electrodes and hence can apply electric stimulation to the brain automatically upon receiving a communication from the control unit.

The processor of the closed-loop system may run programming for assessing the effectiveness of treatment and/or effectiveness of amelioration of a symptom of the neuropsychiatric disorder and modulate a parameter of the treatment as needed without user intervention. Thus, the closed-loop system may not necessarily include a user interface for a user to instruct the stimulation electrode to apply an electrical stimulation to the VC/VS, SGC, or OFC region, or other region to treat the neuropsychiatric disorder in the subject. However, in some embodiments, a user interface may be included in the closed-loop system which may be used to confirm the recommendation of the closed loop system or to override it or to change the recommendation.

In certain aspects, a control algorithm for the methods and systems of the present disclosure may include steps of comparing an electrical signal from a region of the brain of a subject to a normal or reference electrical signal, wherein when the electrical signal is significantly different from the normal or reference electrical signal, the control algorithm includes steps of directing a device to apply electrical stimulation to the brain of the subject, followed by measurement of electrical signal from the region of the brain and comparing it to a normal or reference electrical signal, where when the measured signal is significantly different from normal or reference electrical signal, the algorithm includes step of applying another electrical stimulation to the brain.

In some embodiments, the control algorithm utilizes a machine learning algorithm to analyze inputted brain electrical activity data to automate detection of brain activity features that distinguish symptom states of a neuropsychiatric disorder that are severe enough to need treatment from symptomless states (baseline) or minor symptom states that do not need treatment. The control algorithm then directs a device to apply electrical stimulation to the brain of the subject if the brain activity features indicate a symptom state severe enough that the subject should be treated with electrical stimulation. For example, a machine learning algorithm may be used to correlate the levels of overall power, or power in specific frequency ranges (e.g., alpha, delta, beta, gamma, and/or high gamma) with symptom severity and need for treatment with electrical stimulation. In some embodiments, the level of gamma frequency power (such as 30 Hz to 200 Hz) is correlated with symptom severity to determine if a patient is in need of treatment with electrical stimulation. In some embodiments, electrical stimulation is delivered when the level of an electrical signal that is detected exceeds a set threshold level.

Components of systems for carrying out the presently disclosed methods are further described in the examples below.

### Utility

The methods and systems of the present disclosure find use in a variety of different applications, including the treatment of neuropsychiatric conditions that affect mood and/or behavior of a person suffering from the conditions. Neuropsychiatric disorders that may be treated using the methods and systems presented herein include Major Depressive Disorder (MDD), Generalized Anxiety Disorder (GAD), Post-Traumatic Stress Disorder (PTSD), Addiction, Anorexia, Obsessive-Compulsive Disorder (OCD), Bipolar Disorder (BD) and chronic pain.

In certain aspects, the methods and systems of the present disclosure find use treating a subject suffering from a moderate to severe form of a neuropsychiatric conditions that affect mood and/or behavior of a person suffering from the conditions, for example the subject may be suffering from moderate or severe depression (e.g., Major Depressive Disorder (MDD) or treatment-resistant depression (TRD)), anxiety, addiction, anorexia, OCD, Bipolar Disorder (BD), chronic pain, or PTSD. In certain aspects, a subject may be suffering from a mild form of a neuropsychiatric disorder and may be treated according to the methods or using the systems of the present disclosure to treat the mild disorder and prevent progression into a more severe form.

In certain aspects, the methods and systems of the present disclosure may be used to treat chronic pain, such as one or more symptoms of chronic pain, e.g., constant attention to the experience of pain, emotional toll of the experience of constant pain, depression and/or anxiety. Efficacy of the treatment of patients suffering from chronic pain may be measured in an art accepted manner such as, by a Visual Analog Scale.

Open-loop intracranial stimulation of other brain targets, such as subcallosal cingulate gyrus for MDD, and ventral capsule/ventral striatum or nucleus accumbens for OCD, have failed to show reliable effectiveness in normalizing low or anxious mood in controlled clinical trials. Based on the controlled study described here, closed-loop therapy with electrical stimulation consistently improves mood and/or reduces symptoms of anxiety and/or depression.

Electrical stimulation methods, including implantation of electrodes, are considerably less invasive than targeted ablation approaches. Furthermore, study results show that in examples of acute stimulation, effects on mood state are similarly acute, indicating that the method represents a reversible intervention. This attribute offers the added advantages of (a) precise tuning of therapeutic stimulation to suit the needs of the patient and (b) the incorporation of stimulation into well-controlled clinical trials featuring on/off periods and/or treatment crossover design.

Closed-loop stimulation can be finely targeted and tuned in a personalized manner to achieve more reliable and/or more effective acute symptom relief compared to transcranial stimulation techniques. Likewise, these same attributes of targeting and tuning are more likely to impart neuroplastic change and lasting relief of disease symptoms than transcranial methods, given the ability to deliver considerably more precise stimulation paradigms through intracranial electrodes. The relatively greater precision and tunability of closed-loop stimulation methods has the potential to minimize side effects in individual patients. In addition, unlike transcranial methods, closed-loop stimulation does not require daily or near-daily in-clinic treatment regimens.

Closed-loop stimulation methods consistently show effectiveness in alleviating symptoms of anxiety and depression in affected patients. In addition, stimulation has the potential to be targeted and tuned in a personalized manner that may achieve more reliable and/or more effective symptom relief compared to less targeted peripheral interventions such as vagus nerve stimulation.

Cognitive behavioral therapies and pharmacologic interventions, for example with serotonin-reuptake inhibitors, are among the most established and widely implemented therapeutic approaches for neuropsychiatric disorders, though none has been demonstrated to effect reliable remission in all patients for any given indication. Notably, stimulation presumably achieves its symptom-relieving effects through distinct mechanisms from those enacted by CBT or pharmacologic interventions. Hence, stimulation represents a potentially complementary therapeutic modality that could be combined with CBT and/or pharmacologic treatment to optimize patient benefit.

Similarly, stimulation methods may be optimized through the incorporation of complementary assessment techniques, including physiological and behavioral measures, performance on functionally relevant cognitive tasks, and patient-directed neurofeedback. Moreover, when integrated in real time, these assessment approaches can refine targeting and/or tuning of stimulation and its therapeutic effectiveness.

### Examples of Non-Limiting Aspects of the Disclosure

Aspects, including embodiments, of the present subject matter described above may be beneficial alone or in combination, with one or more other aspects or embodiments. Without limiting the foregoing description, certain non-limiting aspects of the disclosure numbered 1-77 are provided below. As will be apparent to those of skill in the art upon reading this disclosure, each of the individually numbered aspects may be used or combined with any of the preceding or following individually numbered aspects. This is intended to provide support for all such combinations of aspects and is not limited to combinations of aspects explicitly provided below:

1. A closed-loop method for treating a neuropsychiatric disorder in a subject, the method comprising:
mapping a brain of a subject to identify an optimal location in a first brain region for positioning a first electrode for delivery of electrical stimulation to treat the neuropsychiatric disorder;
mapping the brain of the subject to identify an optimal location in a second brain region for positioning a second electrode for detection of an electroencephalographic signal from neural activity associated with a symptom of the neuropsychiatric disorder;
positioning the first electrode at the optimal location in the first brain region to deliver electrical stimulation to the first brain region of the subject;
positioning the second electrode at the optimal location in the second brain region to detect the electroencephalographic signal from the neural activity associated with the symptom of the neuropsychiatric disorder;
detecting the electroencephalographic signal at the second region of the brain of the subject using the second electrode; and
applying electrical stimulation to the first brain region using the first electrode in a manner effective to treat the neuropsychiatric disorder in the subject when the electroencephalographic signal that is detected using the second electrode exceeds a threshold level.

2. The method of aspect 1, further comprising using a control algorithm to automate said applying electrical stimulation when the electroencephalographic signal exceeds the threshold level.

3. The method of aspect 2, wherein the control algorithm comprises a machine learning algorithm.

4. The method of aspect 3, wherein the machine learning algorithm is a supervised machine learning algorithm.

5. The method of any one of aspects 2-4, wherein the control algorithm further modulates one or more programmed stimulation parameters based on the level of the electroencephalographic signal.

6. The method of any one of claims 1-5, further comprising positioning a plurality of electrodes at the second brain region for detection of the electroencephalographic signal by stereoelectroencephalography (sEEG).

7. The method of any one of aspects 1-6, wherein the first brain region comprises a ventral capsule/ventral striatum (VC/VS) region, a subgenual cingulate (SGC) region, or an orbitofrontal cortex (OFC) region of the brain.

8. The method of any one of aspects 1-7, wherein the second brain region comprises a right amygdala region, a left amygdala region, a right orbitofrontal cortex region, a left subgenual cingulate region, or a right hippocampus region.

9. The method of any one of aspects 1-8, wherein the electroencephalographic signal is gamma frequency power.

10. The method of any one of aspects 1-9, wherein the first electrode is placed on a surface of the first brain region or within the first brain region.

11. The method of any one of aspects 1-10, wherein the second electrode is placed on a surface of the second brain region or within the second brain region.

12. The method of any one of aspects 1-11, wherein the first electrode or the second electrode is a non-brain penetrating surface electrode array.

13. The method of any one of aspects 1-12, wherein the first electrode or the second electrode is a brain-penetrating electrode array.

14. The method of any one of aspects 1-13, wherein the neuropsychiatric disorder comprises depression, wherein applying the electrical stimulation treats depression.

15. The method of any one of aspects 1-14, wherein the neuropsychiatric disorder comprises anxiety, wherein applying the electrical stimulation treats anxiety.

16. The method of any one of aspects 1-15, wherein the neuropsychiatric disorder is Major Depressive Disorder (MDD), Generalized Anxiety Disorder (GAD), Post-Traumatic Stress Disorder (PTSD), Addiction, Anorexia, Obsessive-Compulsive Disorder (OCD), or Bipolar Disorder (BD) or chronic pain.

17. The method of any one of aspects 1-16, wherein the electrical stimulation is applied unilaterally or bilaterally.

18. The method of any one of aspects 1-17, wherein the method further comprises assessing effectiveness of the treatment in the subject.

19. The method of any one of aspects 1-18, further comprising monitoring the subject for a period of time, wherein increasing numbers of the electroencephalographic signals exceeding the threshold level detected by the second electrode within a later recording period compared to an earlier recording period indicate increasing severity of the symptom of the neuropsychiatric disorder; and decreasing numbers of electroencephalographic signals exceeding the threshold level detected by the second electrode within a later recording period compared to an earlier recording period indicate decreasing severity of the symptom of the neuropsychiatric disorder.

20. The method of aspect 19, wherein the subject is monitored continuously or intermittently.

21. The method of any one of aspects 1-20, wherein the first brain region and the second brain region are the same or different.

22. A closed-loop method for treating major depressive disorder (MDD) in a subject, the method comprising:
positioning a first electrode to deliver electrical stimulation to a first region of the brain of the subject;
positioning a second electrode to detect a gamma frequency electroencephalographic signal from neural activity associated with MDD symptoms at a second region of the brain of the subject;
detecting the gamma frequency electroencephalographic signal using the second electrode; and
applying electrical stimulation to the first region of the brain of the subject using the first electrode in a manner effective to treat the MDD symptoms in the subject when the gamma frequency electroencephalographic signal that is detected using the second electrode exceeds a threshold level.

23. The method of aspect 22, wherein the first brain region comprises a ventral capsule/ventral striatum (VC/VS) region, a subgenual cingulate (SGC) region, or an orbitofrontal cortex (OFC) region of the brain.

24. The method of aspects 22 or 23, wherein the second brain region comprises a right amygdala, left amygdala region, a right orbitofrontal cortex region, a left subgenual cingulate region, or a right hippocampus region.

25. The method of aspect 23 or 24, wherein said applying electrical stimulation comprises applying bipolar electrical stimulation at the VC/VS region of the brain of the subject.

26. The method of any one of aspects 22-25, wherein said electrical stimulation is applied at a current ranging from 1 mA to 6 mA.

27. The method of any one of aspects 22-26, wherein said electrical stimulation is applied for up to 10 minutes a day.

28. The method of any one of aspects 22-26, wherein said electrical stimulation is applied continuously for a time ranging from 1 hour a day to 24 hours a day.

29. The method of any one of aspects 22-28, further comprising monitoring the subject for a period of time, wherein increasing numbers of gamma frequency electroencephalographic signals exceeding the threshold level detected by the second electrode within a later recording period compared to an earlier recording period indicate increasing severity of MDD symptoms; and decreasing numbers of gamma frequency electroencephalographic signals exceeding the threshold level detected by the second electrode within a later recording period compared to an earlier recording period indicate decreasing severity of MDD symptoms. 30. The method of aspect 29, wherein the subject is monitored continuously or intermittently.

31. A closed-loop method for ameliorating a symptom of a neuropsychiatric disorder in a subject, the method comprising:
mapping a brain of a subject to identify an optimal location in a first brain region for positioning a first electrode for delivery of electrical stimulation to ameliorate the symptom of the neuropsychiatric disorder;
mapping the brain of the subject to identify an optimal location in a second brain region for positioning a second electrode for detection of an electroencephalographic signal from neural activity associated with the symptom of the neuropsychiatric disorder;
positioning the first electrode at the optimal location in the first brain region to deliver electrical stimulation to the first brain region of the subject;
positioning the second electrode at the optimal location in the second brain region to detect the electroencephalographic signal from the neural activity associated with the symptom of the neuropsychiatric disorder;
detecting the electroencephalographic signal at the second region of the brain of the subject using the second electrode; and
applying electrical stimulation to the first brain region using the first electrode in a manner effective to ameliorate the symptom of the neuropsychiatric disorder in the subject when the electroencephalographic signal that is detected using the second electrode exceeds a threshold level.

32. The method of aspect 31, wherein the symptom comprises depression and wherein applying the electrical stimulation ameliorates depression.

33. The method of aspect 32, wherein depression is measured by Visual Analogue Scale for Depression (VAS-D), Hamilton Depression Rating Scale (HAM-D). Montgomery-Asberg Depression Rating Scale (MADRS), or Beck Depression Inventories (BDI) score and applying the electrical stimulation is effective in reducing symptom severity according to the VAS, HAM-D, MADRS, or BDI score.

34. The method of aspect 31, wherein the symptom comprises anxiety and wherein applying the electrical stimulation ameliorates anxiety.

35. The method of aspect 34, wherein anxiety is measured by Visual Analogue Scale for Anxiety (VAS-A) or Beck Anxiety Inventories (BAT) score and applying the electrical stimulation is effective in reducing anxiety according to the VAS-A or the BAI score.

36. The method of any one of aspects 31-35, wherein the first electrode is placed on a surface of the first brain region or within the first brain region.

37. The method of any one of aspects 31-36, wherein the second electrode is placed on a surface of the second brain region or within the second brain region.

38. The method of any one of aspects 31-37, wherein the first electrode or the second electrode is a non-brain penetrating surface electrode array.

39. The method of any one of aspects 31-38, wherein the first electrode or the second electrode is a brain-penetrating electrode array.

40. The method of any one of aspects 31-39, wherein the neuropsychiatric disorder is Major Depressive Disorder (MDD), Generalized Anxiety Disorder (GAD), Post-Traumatic Stress Disorder (PTSD), addiction, anorexia. Obsessive-Compulsive Disorder (OCD), or Bipolar Disorder (BD), or chronic pain.

41. The method of any one of aspects 31-40, wherein the method further comprises assessing effectiveness of the treatment in the subject.

42. The method of any one of aspects 31-41, further comprising using a control algorithm to automate said applying electrical stimulation when the electroencephalographic signal exceeds the threshold level.

43. The method of aspect 42, wherein the control algorithm comprises a machine learning algorithm.

44. The method of aspect 43, wherein the machine learning algorithm is a supervised machine learning algorithm.

45. The method of any one of aspects 42-44, wherein the control algorithm further modulates one or more programmed stimulation parameters based on the level of the electroencephalographic signal.

46. The method of any one of aspects 31-45, further comprising positioning a plurality of electrodes at the second brain region for detection of the electroencephalographic signal by stereoelectroencephalography (sEEG).

47. The method of any one of aspects 31-46, wherein the first brain region comprises a ventral capsule/ventral striatum (VC/VS) region, a subgenual cingulate (SGC) region, or an orbitofrontal cortex (OFC) region of the brain.

48. The method of any one of aspects 31-47, wherein the second brain region comprises aright amygdala region, a left amygdala region, aright orbitofrontal cortex region, a left subgenual cingulate region, or a right hippocampus region.

49. The method of any one of aspects 31-48, wherein the electroencephalographic signal is gamma frequency power.

50. A system for treating a neuropsychiatric disorder in a subject, the system comprising:
a stimulation electrode adapted for positioning at a first region of the brain of the subject;
a detection electrode adapted for positioning at a second region of the brain of the subject and for recording an electroencephalographic signal from the second region of the brain of the subject; and
a processor programmed to instruct the stimulation electrode to apply an electrical stimulation to the first region of the brain in a manner effective to treat the neuropsychiatric disorder in the subject when an electroencephalographic signal that exceeds a threshold level is detected using the second electrode.

51. The system of aspect 50, wherein the first brain region comprises a ventral capsule/ventral striatum (VC/VS) region, a subgenual cingulate (SGC) region, or an orbitofrontal cortex (OFC) region of the brain.

52. The system of aspect 50 or 51, wherein the second brain region comprises a right amygdala region, a left amygdala region, a right orbitofrontal cortex region, a left subgenual cingulate region, or a right hippocampus region.

53. The system of any one of aspects 50-52, wherein the electroencephalographic signal is gamma frequency power.

54. The system of any one of aspects 50-53, wherein the stimulation electrode or the detection electrode is anon-brain penetrating electrode array.

55. The system of any one of aspects 50-54_wherein the stimulation electrode or the detection electrode is a brain penetrating electrode array.

56. The system of any one of aspects 50-55, wherein the neuropsychiatric disorder comprises depression, wherein applying electrical stimulation treats the depression.

57. The system of any one of aspects 50-56, wherein the neuropsychiatric disorder comprises anxiety, wherein applying electrical stimulation treats the anxiety.

58. The system of any one of aspects 50-57, wherein the system further comprises a user interface comprising an input electronically coupled to the processor for instructing the stimulation electrode to apply an electrical stimulation to the first region of the brain to treat the neuropsychiatric disorder in the subject.

59. The system of aspect 58, wherein the user interface is password protected and is operable by a health care practitioner.

60. The system of any one of aspects 50-59, wherein the neuropsychiatric disorder is Major Depressive Disorder (MDD), Generalized Anxiety Disorder (GAD), Post-Traumatic Stress Disorder (PTSD), Addiction, Anorexia, Obsessive-Compulsive Disorder (OCD), or Bipolar Disorder (BD), or chronic pain.

61. The system of any one of aspects 50-60, wherein the processor is further programmed to modulate one or more programmed stimulation parameters according to the algorithm's control law; and apply the modulated electrical stimulation to the first region of the brain via the stimulation electrode in a manner effective to treat the neuropsychiatric disorder.

62. The system of any one of aspects 50-61, wherein the processor is further programmed to set a maximum number of electrical stimulations per day.

63. The system of any one of aspects 50-62, wherein the processor is further programmed to set a total amount of time of electrical stimulation per day.

64. A system for ameliorating a symptom of a neuropsychiatric disorder in a subject, the system comprising:
a stimulation electrode adapted for positioning at a first region of the brain of the subject;
a detection electrode adapted for positioning at a second region of the brain of the subject and for recording an electroencephalographic signal from the second region of the brain of the subject; and
a processor programmed to instruct the stimulation electrode to apply an electrical stimulation to the first region of the brain in a manner effective to ameliorating the symptom of the neuropsychiatric disorder in the subject when an electroencephalographic signal that exceeds a threshold level is detected using the detection electrode.

65. The system of aspect 64, wherein the first brain region comprises a ventral capsule/ventral striatum (VC/VS) region, a subgenual cingulate (SGC) region, or an orbitofrontal cortex (OFC) region of the brain.

66. The system of aspect 64 or 65, wherein the second brain region comprises a right amygdala region, a left amygdala region, a right orbitofrontal cortex region, a left subgenual cingulate region, or a right hippocampus region.

67. The system of any one of aspects 64-66, wherein the electroencephalographic signal is gamma frequency power.

68. The system of any one of aspects 64-67, wherein the symptom comprises depression and wherein applying the electrical stimulation ameliorates depression.

69. The system of any one of aspects 64-68, wherein the symptom comprises anxiety and wherein applying the electrical stimulation ameliorates anxiety.

70. The system of any one of aspects 64-69, wherein the stimulation electrode or the detection electrode is a non-brain penetrating electrode.

71. The system of any one of aspects 64-70, wherein the stimulation electrode or the detection electrode is a brain penetrating electrode.

72. The system of any one of aspects 64-71, wherein the system further comprises a user interface comprising an input electronically coupled to the processor for instructing the stimulation electrode to apply an electrical stimulation to the first region to ameliorate the symptom of the neuropsychiatric disorder in the subject.

73. The system of aspect 72, wherein the user interface is password protected and is operable by a health care practitioner.

74. The system of any one of aspects 64-73, wherein the neuropsychiatric disorder is Major Depressive Disorder (MDD), Generalized Anxiety Disorder (GAD), Post-Traumatic Stress Disorder (PTSD). Addiction, Anorexia, Obsessive-Compulsive Disorder (OCD), or Bipolar Disorder (BD), or chronic pain.

75. The system of any one of aspects 64-74, wherein the processor is further programmed to modulate one or more programmed stimulation parameters according to the algorithm's control law; and apply the modulated electrical stimulation to the first region of the brain via the stimulation electrode in a manner effective to ameliorate the symptom of the neuropsychiatric disorder.

76. The system of any one of aspects 64-75, wherein the processor is further programmed to set a maximum number of electrical stimulations per day.

77. The system of any one of aspects 64-76, wherein the processor is further programmed to set a total amount of time of electrical stimulation per day.

### EXAMPLES

As can be appreciated from the disclosure provided above, the present disclosure has a wide variety of applications. Accordingly, the following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, dimensions, etc.) but some experimental errors and deviations should be accounted for. Those of skill in the art will readily recognize a variety of noncritical parameters that could be changed or modified to yield essentially similar results.

### Example 1: CLOSED-LOOP NEUROMODULATION FOR TREATMENT OF RESISTANT DEPRESSION

Major depressive disorder (MDD) is a common and disabling disorder with a high rate of treatment resistance¹. Intracranial brain stimulation has shown promise as a treatment for refractory MDD, but inter-individual response heterogeneity has contributed to inconsistent findings in definitive clinical trials of deep brain stimulation²⁻⁴. Previous approaches have targeted single structures in the brain in all subjects with largely fixed, constant stimulation dosing. This open-loop approach has been successful in Parkinson's disease⁵ and epilepsy⁶. In MDD, however, multiple potential anatomical stimulation targets have been identified⁷, and none have proven sufficiently promising for durable treatment response. Evidence that different neural circuits appear to underlie different subsets of symptoms experienced by MDD patients speaks for the need of personalized circuit targeting⁸.

Personalized temporal control over treatment delivery may further compliment personalized circuit targeting. In closed-loop neuromodulation, a patient's own physiological activity is used to selectively trigger stimulation only when a pathologic state is detected⁹. This approach may provide better benefit over open-loop therapy because it delivers therapy in a way that addresses the underlying circuit dysfunction and varies in response to ongoing dynamics in the state of neural networks. Because mood-related effects of neuromodulation have been shown to be state-dependent^{10,11}, this specificity may be critical for success in MDD patients who have frequent state-changes. Furthermore, closed-loop stimulation mitigates the concern for reduced therapeutic effect due to neural adaptation¹², and preserves battery life and reduces side-effects by reducing intervention time. However, a symptom-specific biomarker is a precondition for closed-loop therapy and has not previously been identified in MDD. Here we report our experience with a patient, where we identify a biomarker of MDD symptoms during a 10-day period of intracranial corticolimbic circuitry mapping. We then successfully implement the biomarker in an implanted closed-loop neuromodulation therapy. This is the first demonstration of chronic closed-loop neuromodulation in a psychiatric disorder. Our approach could be broadly applied to any network disorder.

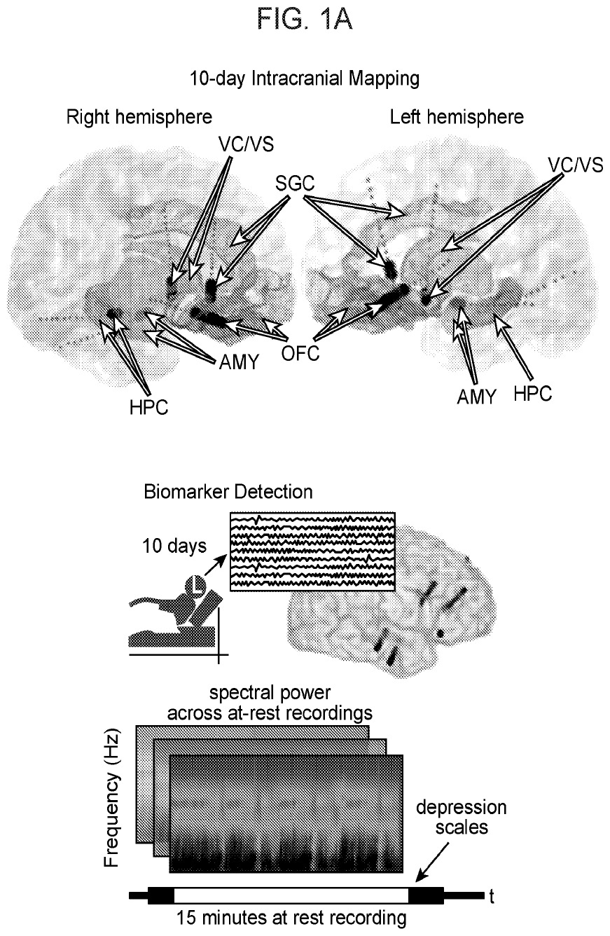

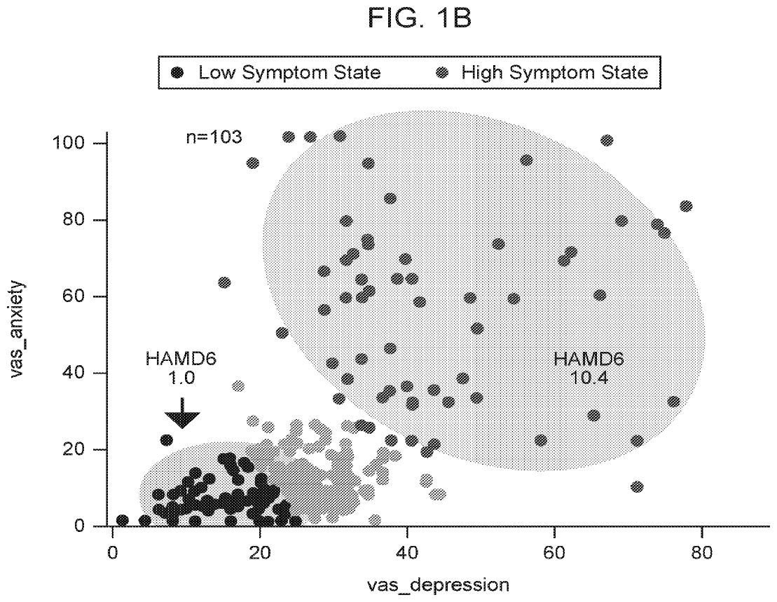

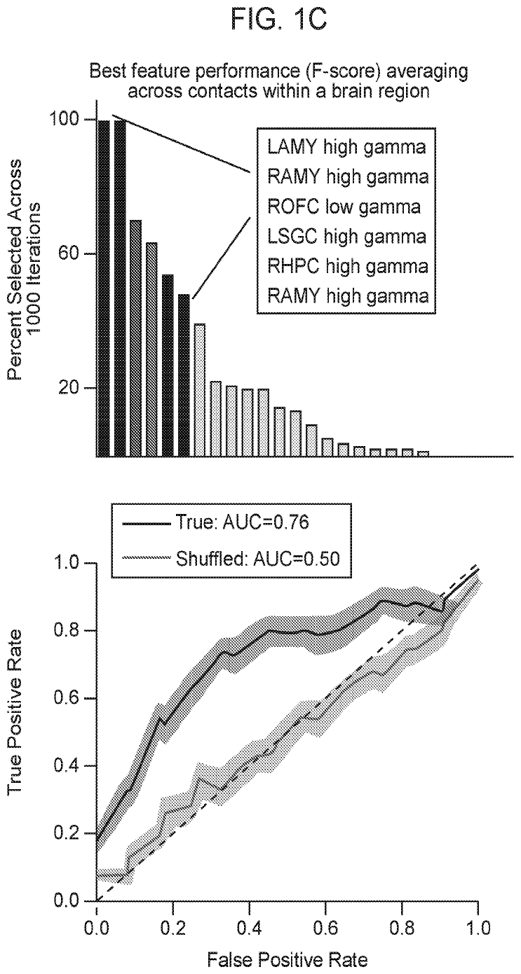

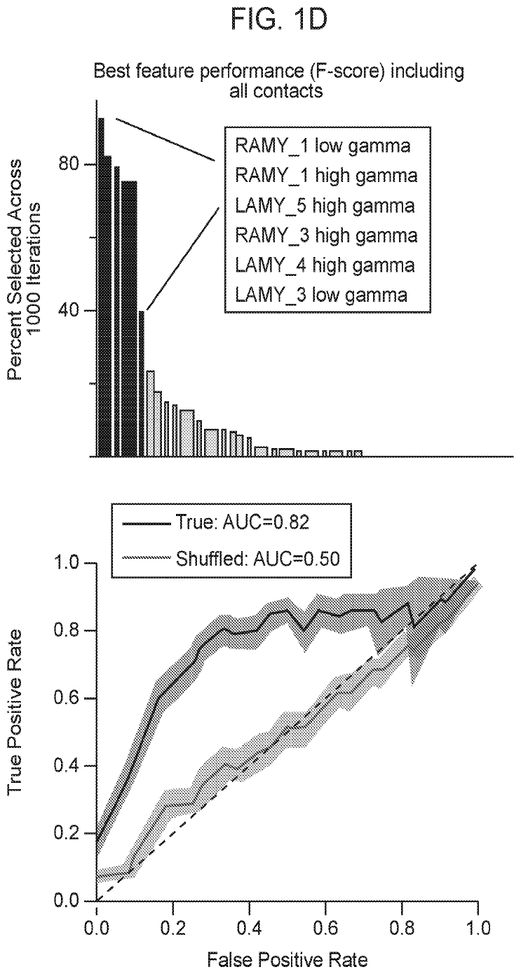

The patient was a 36-year-old woman with childhood onset severe treatment resistant MDD (Montgomery Asberg Depression Rating Scale: 36/54) unresponsive to four antidepressant medications, augmentation strategies, electroconvulsive therapy, and transcranial magnetic stimulation who participated in a clinical trial of personalized closed-loop neurostimulation (Presidio trial, see Scangos et al. 2021 for additional case history¹⁰). In the first trial stage, we performed. We found dimensionally restrictive clinical responses to stimulation across this corticolimbic stimulus-response mapping of the patient's emotion circuitry employing 10 stereoelectroencephalography (sEEG) electrodes implanted bilaterally in the orbitofrontal cortex (OFC), amygdala (AMY), hippocampus (HPC), ventral capsule/ventral striatum (VC/VS), and subgenual cingulate (SGC)¹³⁻¹⁷ circuitry that aligned with different types of depression symptoms¹⁰. Neural activity was continuously recorded for 10 days while the patient performed 335 visual analog scales of depression (VAS-D) and anxiety (VAS-A)¹⁸ and underwent multiple Hamilton Depression Rating Scale assessments (6-item version [HAMD6])¹⁹. Scores were distributed across the full range of the patient's natural emotions (VAS_A: mean 17.5, SD 21.7; VAS_D: mean 26.3, SD 12.9). We used these measures to define two symptom states (high and low symptom severity. FIGS. 1A-1B).

We then identified the 15-minutes of resting state sEEG activity that coincided with the clinical measures where one of the two states was present (n=35). We identified the sEEG spectral activity features that discriminated high and low symptom states employing six standard frequency bands and two cross-validated supervised machine learning models at different spatial resolutions. In the first model, spectral power was averaged across all contacts within brain regions (60 features). We found gamma power in the bilateral AMY, right OFC, left SGC, and right HPC had high state discriminative potential (Accuracy: mean 0.73, std 0.08; AUC: mean 0.76, std 0.10) (FIG. 1C). The second model, where the feature set was defined by spectral power for each electrode contact (246 features), allowed us to account for intraregional variability due to electrode positioning. We found bilateral AMY gamma power alone was sufficient for detecting the high symptom severity state (Accuracy: mean 0.77, std 0.09; AUC: mean 0.82, std 0.11, FIG. 1D). Together, the findings suggested AMY gamma power as a biomarker of the highly depressed state.

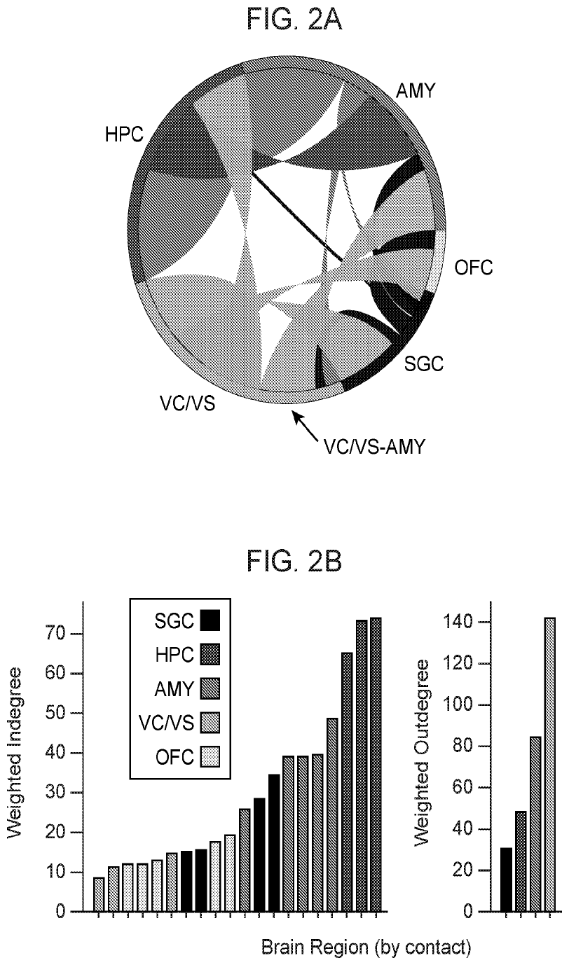

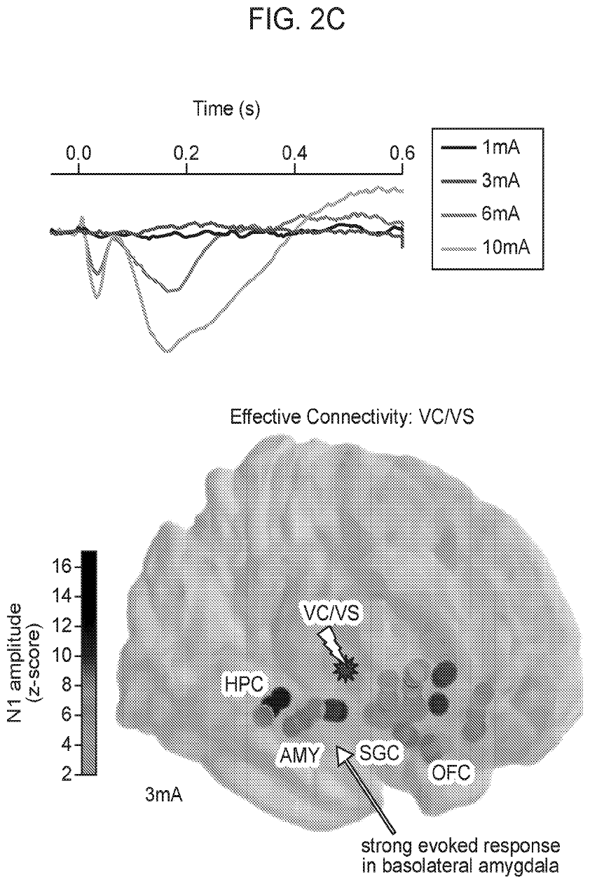

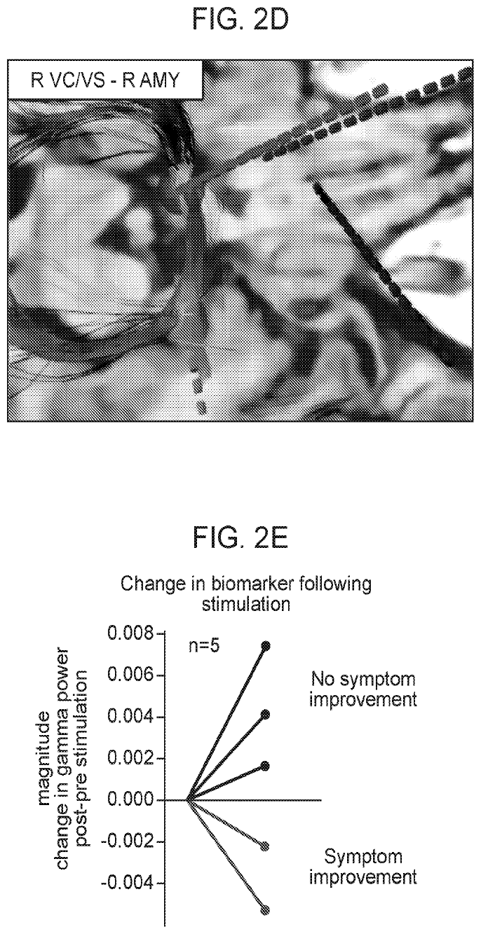

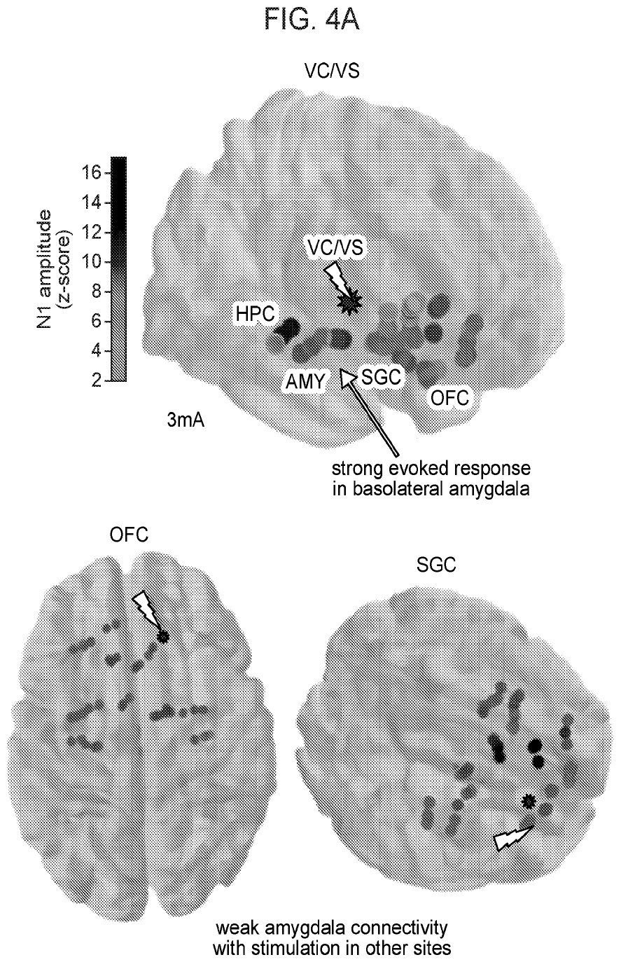

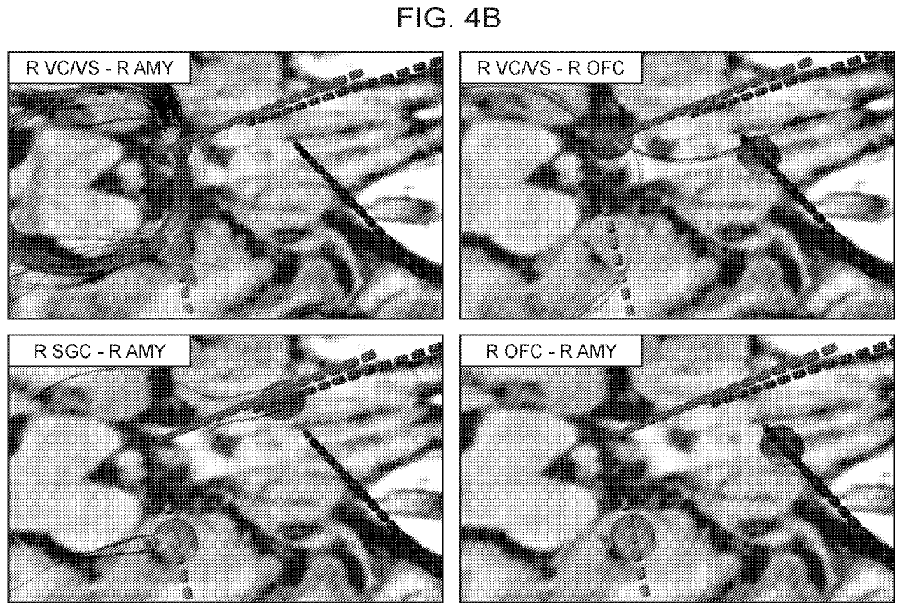

We identified the right VC/VS as the brain site in this patient where electrical stimulation led to consistent improvement of depression symptoms in a sustained, dose-dependent manner¹⁰. We next examined the connectivity across the corticolimbic network to determine whether right hemispheric VC/VS and AMY nodes constituted a structurally and functionally connected subnetwork so that VC/VS stimulation directly influenced AMY biomarker activity. To examine effective network connectivity, we delivered brief stimulation pulses across adjacent contacts in one brain region and examined the evoked potential (EP) in distant contacts to generate a global directed network graph²⁰ (FIG. 2A). We then quantified the relative importance of nodes in this graph²¹. We found AMY and HPC were sites that integrated signals from distant brain regions (highest weighted indegree, FIG. 2B left). The VC/VS was a site that strongly influenced many distant brain regions (highest weighted outdegree, FIG. 2B right). Furthermore, we observed a strong AMY-evoked response to VC/VS stimulation (FIG. 2c), that was comparatively stronger than other network nodes (FIG. 4A). We examined the putative axonal fiber tracts that might mediate the effective connectivity using deterministic tractography with diffusion tensor imaging and found that there was also dense structural connectivity between the right VC/VS and AMY comprised of two fiber tracts, the stria terminalis and the ansa peduncularis (FIG. 2D, FIG. 4B). Finally, VC/VS stimulation at contacts 2+/3− led to a clinical improvement in symptom severity on 11/13 trials. We examined the change in AMY gamma power as a result of this stimulation on trials where we had pre- and post-VAS scales and resting-state sEEG activity (n=5 trials). We observed a reduction in AMY gamma power on both trials where symptom severity improved (VAS-D pre-post: 29-20, 27-21; VAS-A pre-post: 4-0, 13-8; FIG. 2E), which was absent on trials when symptom severity worsened due to an alternate stimulation paradigm (VAS-D: 14-76, VAS-A: 4-77), or remained steady due to minimal symptoms at baseline (VAS-D: 16-17, VAS-A: 6-4) or an primary increase in energy over depression (VAS-D 30-31; VAS-A: 8-13; VAS-E 7-87). The effective and structural network mapping and spectral-power biomarker discovery together suggest that AMY-VC/VS may represent an important limbic subnetwork in this patient that both reflects depression severity and can be modulated to decrease clinical symptoms.

We next implanted the FDA-approved and commercially available NeuroPace RNS® System²² unilaterally in the right hemisphere (FIG. 3A), with two separate sensing/stimulation four-contact depth leads placed in the AMY and VC/VS, respectively (FIG. 3B). The RNS System neurostimulator continuously senses neural activity, and detection of pre-specified patterns of activity are coupled to delivery of electrical stimulation [trains of charge-balanced biphasic square-wave pulses] to designated electrodes. We assessed whether we could replicate the symptom-specific biomarker for closed-loop control using right AMY recordings. Using the NeuroPace RNS System telemetry wand to stream wireless real-time electrocorticography in the laboratory, we recorded 10-minute trials of continuous AMY activity while the patient engaged in naturalistic activities (e.g. social media, talking to family on the phone, watching movies, virtual therapy appointment) and paired the neural activity with clinical severity ratings (FIG. 3C). Again, we found that AMY gamma power was highly correlated with both the VAS-D (r²: 0.82, p: 3.4e-05), VAS-A (r²: 0.52, p: 0.01), and HAMD-6 (r²: 0.73, p: 4.3e-04. FIG. 3D) and significantly differentiated the high symptom severity state from the low symptom severity state (ANOVA, F:8.33, p=0.015).

The clinical effect of right VC/VS stimulation was also replicated. Bipolar stimulation at the VC/VS contacts that engaged the stria terminalis and the ansa peduncularis (VC/VS contacts 2+/3−, 3+/4, 100 Hz, 120 us, 1-2 mA) led to an acute dose-dependent symptom improvement across the majority of trials, and the effect was strongest, and preferred by the patient (described as pleasurable and energizing) at the predicted location (VC/VS contacts 3+/4−) in comparison to other VC/VS and AMY contact pairs (FIG. 3E). Furthermore, we found that over 15 and 30 minutes, 6 seconds of intermittent stimulation at 1 mA was clinically effective and below the patient's perceptual threshold so that ON/OFF states would not affect normal daily functioning (FIG. 3F, left). Increasing the dose to 2 mA or changing contacts led the patient to perceive stimulation and worsened the clinical response on some trials (FIG. 3F, right).

We then implemented the closed-loop therapy such that 6 seconds of stimulation would be delivered following the device's automated detection of the biomarker (FIG. 3G). The NeuroPace System uses a half-band detector to estimate power in specific frequency bands. We calibrated the device to continuously sense the AMY gamma biomarker. A ‘detection’ was triggered when gamma power crossed a threshold of 0.8% of full amplitude scale assessed within a 2 second sliding window for AMY contacts ¾ or ½. We found that the number of detections within 10-minute recording periods was 87% predictive of symptom severity state and that the detection number was highly correlated with VAS-D (r²: 0.59, p: 1.2e-04), VAS-A (r²: 0.52, p: 4.6e-04), and HAMD-6 (r²: 0.65, p: 1.98e-05, FIG. 3H). Over the course of 2 months, we found that on average, there were 468 detections per day (SD: 206) distributed across the day with minimal stimulations during the night. We set a stimulation cap of 300 therapies/day (30 minutes of total stimulation) so as not to disturb sleep from evening therapy.

Implementation of closed-loop therapy led to a rapid improvement in both symptom severity (measured daily with HAMD-6, VAS scales) and depression (measured periodically with the MADRS scale). While these findings show closed-loop stimulation can achieve the intended outcome, an adequately powered, randomized controlled trial with blinding will be needed to determine its efficacy. The subject's MADRS score dropped from a 33 prior to turning treatment ON to a 14 (58% reduction) at the first during-treatment assessment carried out after 12 days of stimulation and dropped below 10 (remission) several months later. Similarly, her HAMD-6 and VAS_D scores dropped precipitously the morning after stimulation was started (HAMD6: 12.0 to 1.0; VAS_D: 77 to 23) and were lower the week after stimulation was turned ON in comparison to the previous week (HAMD6: mean 16 (SD 2.82) to 1.6 (SD 1.60), Welch's t-test p=0.08; VAS-D: 77.33 (14.57) to 10.48 (5.74); Welch's t-test, p=0.02, FIG. 3I).

To further evaluate whether our defined closed-loop algorithm triggered the delivery of therapy in a manner linked to the patient's symptoms, and not randomly, we examined the relationship between a morning symptom severity rating and the daily detection count over two months. Because morning symptom severity ratings may reflect symptom state over a variable length of time and thus, may be mismatched in either amplitude or phase with the neural biomarker detected by the device, we used dynamic time warping (DTW) to first non-linearly align daily symptom severity (VAS-D) and biomarker detection count time-traces and next calculate their relative post-alignment distance²³. We found that fluctuation in daily symptoms was significantly associated with the fluctuation in number of biomarker events detected by the device (p=2.8e-04, FIG. 3J). These results suggest that our biomarker detection algorithm detects change in symptom severity significantly better than random chance and provides initial evidence that the RNS System is functioning in a manner where dysfunctional circuit activity underlying MDD symptoms informs therapy delivery in closed-loop fashion.

### Discussion

This is to our knowledge the first instance of the successful development of a personalized biomarker of depression-specific symptoms and implementation of closed-loop therapy for MDD. Success was predicated on a clinical mapping trial prior to chronic device placement, a strategy that has been utilized in epilepsy to map seizure foci in a personalized manner²⁴ but has not previously been performed in other neuropsychiatric conditions. We developed a comprehensive multimodal framework for selection of sensing and stimulation brain targets during this multiday mapping period (FIG. 5). Our approach included personalized stimulus response mapping, pairing of resting-state signals with clinical symptom measures, and identification of functionally and structurally connected subnetworks across the corticolimbic network.

We found that AMY gamma power alone was sufficient to predict a pathologic high symptom severity state and that VC/VS stimulation lowered AMY gamma power in conjunction with improving MDD symptoms. Prior work supports our findings that AMY plays an important role in the processing and mediation of emotion^{25,26}, that the VC/VS and AMY are important nodes within mood regulatory circuitry²⁷ and that bursts of high frequency activity within the AMY are correlated with emotion states¹⁵. The mechanism underlying the relationship of AMY activity to the depressed state and how VC/VS stimulation might ameliorate this state and normalize AMY activity remains unknown. However, the VC/VS region, a node in the reward pathway, is one of the most thoroughly studied DBS targets for depression¹³ and may be particularly effective for treating anhedonia's. In the face of emotionally negative input, stimulation of the VC/VS may reduce AMY hyperactivity both by driving underactive direct striatal connections, and through dopamine-mediated interactions within the reward pathway, which could in turn influence the frontally mediated appraisal of the emotional input. The successful reduction in AMY gamma may be a requirement for improving symptoms. Research will be needed to determine the degree to which this AMY biomarker and the AMY-VS/VS subcircuit are present over time within individuals, and whether they underlie depression in general or are specific to this patient or a subset of patients with MDD. Our methodology was designed to address this variability and enables us to identify an individualized stimulation target and neural biomarker for each subject. Based on prior studies, we expect the SGC to be another important brain stimulation target in a subset of subjects^{29,30}. Group level analyses may determine that certain symptom profiles are predictive of a stimulation target and help define symptom state thresholds that enhance biomarker identification.

We also identified a set of detection algorithms using the NeuroPace RNS System that were able to detect AMY gamma power despite device capabilities designed for seizure detection. Whether this ability is restricted to high-frequency activity remains unknown due to our restricted ability to explore other frequency bands because of device limitations. We were only able to determine that alpha frequency power may also correlate with symptom severity but was not of utility for detecting mood state in this patient. New device capabilities that sense continuous neural activity and have the capability to integrate information on the time-scale of minutes may improve our ability to incorporate more complex biomarkers into closed-loop control³¹. While traditional DBS is performed bilaterally, the NeuroPace device was implanted on the right side alone based on the clinical mapping study and safety considerations. The reasons for the hemispheric differences we observed are unknown but we speculate that they could be due to lateralization of emotion regulation^{32,33} or electrode placement within the VC/VS bilaterally.

We conceptualized MDD as a dynamic process in which symptoms arise when a dysfunctional activity state emerges in one or more pre-frontal/limbic brain networks subserving mood-related functions. In this model stimulation has an immediate impact on symptom severity. By repeatedly improving symptoms as they emerge over time, an antidepressant effect is achieved. An acute response to VC/VS stimulation, as we repeatedly observed in this subject, has been observed in prior studies in the intraoperative or programming periods^{34,35} yet is not always sustained once continuous stimulation is initiated. By delivering intermittent stimulation in a closed-loop manner we hypothesize that we are able to repeatedly obtain these acute effects and employ them as a means to treat depression. In line with our model, this patient's depression acutely improved after closed-loop therapy was initiated and the effect was sustained. Both the rapidity and intensity of the clinical response is highly unusual in treatment resistant depression, where the 1-year remission rate for ‘treatment as usual’ is ˜3.5%³⁶ and symptom relief from DBS can take months to emerge². The intent of this report was not to address the efficacy of closed-loop neuromodulation for MDD which would require a double-blind, randomized controlled assessment carried out in a group of patients large enough to provide sufficient power to detect clinically meaningful effects. Here we establish proof-of-concept for a new powerful approach for the treatment of neuropsychiatric disorders and show its feasibility in a single patient. Whether or not our methodology is capable of identifying subcircuits associated with biomarker/stimulus pairs that can be implemented as closed-loop therapy across individuals is an important area for further investigation that will determine the promise of this approach.

Altogether, our findings suggest that quantitative circuit-based treatment development is possible. The novel framework presented in this report could advance biomarker-based neural interfaces and enhance mechanistic understanding and treatment of a broad range of neuropsychiatric conditions.

### Example 2: Methods

### Surgical Procedure

The patient gave written informed consent for participation in a clinical trial of closed-loop neuromodulation for treatment resistant major depressive disorder (MDD) (Presidio: clinicaltrials.gov/ct2/show/NCT04004169), approved by the Institutional Review Board at the University of California, San Francisco and by the U.S. Food and Drug Administration (FDA). Please see the Life Sciences Reporting Summary published along with this case report for additional details. The patient underwent two implant surgeries. In the first, we surgically implanted ten stereoelectroencephalography (sEEG) electrodes (PMT Corporation, Chanhassen, MN) within the orbitofrontal cortex (OFC), amygdala (AMY), hippocampus (HPC), ventral capsule/ventral striatum (VC/VS), and subgenual cingulate (SGC) as described in Scangos et al. 2021¹⁰. Exploratory intracranial stimulation and recording took place over a 10-day period (October 2019). After 10 days the electrodes were explanted. In the second implant surgery, we implanted the NeuroPace RNS System with two four-contact depth leads (30-cm lead length, 3.5-mm electrode spacing) in the right VC/VS and AMY, guided by our findings in the 10-day recording and monitoring (mapping) period. Surgical targeting was planned in Brainlab iPlan Cranial Software using DTT³⁷ or coordinate-based targeting³ in accordance with published work. Computerized tomography (CT) was used intraoperatively to confirm electrode placement. No complications of surgery occurred.

### Clinical Measures

To assess moment-to-moment changes in MDD symptom severity we used visual analog scales³⁸ (VAS) of depression (VAS-D) and anxiety (VAS-A) and the HAMD6 subscale of the HAMD-17 which is thought to capture the core symptoms of the full-scale and has been used to assess the rapid effects of antidepressants^{19,39}. Our symptom assessment strategy included an a priori plan to consider dimensions of depression that can change in the course of a day as represented in the HAMD6 subscale of the HAMD-17, which includes: Q1) sadness, Q2) guilt, Q3) apathy. Q4) fatigue, Q5) anxiety, and Q6) energy but focus only on the dimensions that were possible to meaningfully operationalize in the setting of repeated testing with a VAS (depression, anxiety) and were the smallest number needed to reflect the symptom profile of the patient. Study data v ere collected and managed using REDCap electronic data capture tools hosted at UCSF^{40,41}. REDCap (Research Electronic Data Capture) is a secure, web-based software platform designed to support data capture for research studies, providing 1) an intuitive interface for validated data capture; 2) audit trails for tracking data manipulation and export procedures; 3) automated export procedures for seamless data downloads to common statistical packages; and 4) procedures for data integration and interoperability with external sources. The patient performed her in-lab and at-home surveys using this tool on a study iPAD. Once the NeuroPace device was in place she was asked to perform at-home surveys 2 to 3 times per day.

### Signal Processing:

Intracranial EEG (iEEG) recordings derived from sEEG electrodes were used to derive measures of activity and connectivity. This is a high-resolution intracranial recording technique^{42,43} and is commonly used in the pre-surgical evaluation of drug-resistant epilepsy. Stimulation through these electrodes (single pulses⁴⁴ or continuous stimulation¹⁴) was used to perform stimulus-response mapping, assess connectivity and assess the effects of perturbation to MDD networks. Data acquisition of iEEG recordings and evoked potential (EP) mapping were performed with a 256 channel Nihon Kohden clinical system and secondary data stream at a sampling rate of 10 kHz. Standard sEEG pre-processing techniques were conducted in Python and involved application of a 2-250 Hz bandpass filter, notch filters at line noise frequency and harmonics, down sampling to 512 Hz, and common average referencing across all channels. Signal processing vas performed using the continuous wavelet transformation (Morlet, 6-cycles)⁴⁵ in 30 s intervals to obtain power in 6 spectral frequency bands (delta=1-4 Hz, theta=5-8 Hz, alpha=9-12 Hz, beta=13-30 Hz, low gamma=31-70 Hz, high gamma=71-150 Hz). Artifacts in EP analysis were removed with 8^{th} order Butterworth filter.

### Electrode Stimulation

Our approach to clinical mapping is discussed elsewhere¹⁰. Briefly, we tested the clinical effect of a set of stimulation parameters used in the evaluation of patients with epilepsy (1 or 100 Hz, 100 us, 1-6 mA) through a systematic bipolar stimulation survey and then blinded sham-controlled stimulation studies across 10 days. Where indicated, brain stimulation configuration is represented by contact number and polarity (ex. 2+/3− reflects that contact 2 is anode, contact 3 is cathode). Once the NeuroPace device was in place, we repeated the bipolar stimulation survey and sham controlled stimulation using the same clinical scales as outcome measures. The patient was blinded to stimulation location and parameters. If the patient experienced non-affective clinical sensation from stimulation, such as facial pulling, the stimulation intensity was lowered. Closed-loop neuromodulation introduces a new stimulation parameter—the burst duration delivered once a depressed state is detected. We tested the effect of burst duration in a systematic manner by delivering increasing durations of stimulation (6, 12, 18, 36 s bursts, 100 Hz, 120 us, 1 mA) while keeping total stimulation time constant over 15 and 30 minute time intervals. Our strategy was to identify a set of parameters where intermittent stimulation was clinically effective over a time interval but each burst onset was not detected by the patient to prevent interfering with normal activities, and to select shorter more frequent bursts over less frequent but longer stimulation periods in order to increase specificity.

### Biomarker Discovery

The development of symptom-specific biomarkers in depression have been challenged by the lack of objectively observable clinical symptoms such as those that exist in movement disorders, and the multi-dimensional nature of MDD. We addressed this challenge during the mapping trial in several ways. We included a high trial count to address inherent noise in the self-assessment of internal state. We also ensured there was variability across time in the measures by including the presence of mood variability as one of the inclusion criteria of the clinical trial, and having the patient perform naturalistic activities in the laboratory setting that induced symptom state variation (e.g. recalling life events, watching movies, social media) during the 10-day period. Then, based on the anxiety and depression dimensions of the VAS scale, we sought to identify states of high symptom severity and low symptom severity objectively using semi-supervised k-means clustering. Specifically, to discriminate noisy ratings from clinically-relevant ratings we used cluster tuning and cluster aggregation to discriminate two symptom severity states based on VAS ratings that best corresponded to the clinically established HAMD6 scores.

To identify spectral biomarkers related to symptom severity state, we asked the patient to rest for 15-minutes and then assessed MDD symptom severity (self-report HAMD6. VAS scales) about five times each day. These 15-minute intervals were required to be at least 1 hour from stimulation to minimize any potential influence of stimulation on the neural activity. Spectral power was calculated in 30 s intervals and averaged across the 15 min recording period to obtain power in six frequency bands. We then modeled differences in spectral power across symptom states to identify spectral power features that were predictive of high symptom severity using cross-validated machine learning models. In the first model, the feature set was defined by spectral power averaged across all contacts within brain regions (10 regions×6 bands=60 features). In a second model, the feature set was defined by spectral power for each electrode contact (41 anatomically verified contacts×6 bands=246 features). The modeling pipeline consisted of feature selection based on ANOVA F value and classification using penalized logistic regression trained on 80% of the data and tested on the remaining 20%. We repeated the train/test schema 1000 times to ensure stability in AUC score and feature importance histograms. Significance was assessed by comparing the output of our logistic classifier to a null model obtained from randomly permuting class labels 1000 times.

In closed-loop mode, delivery of therapeutic stimulation is expected to normalize the biomarker that triggers the stimulation. In order to test the effect of VC/VS stimulation on biomarker activity, we examined the change in gamma power in two 30 s intervals before and after a period of continuous stimulation. We included all types of stimulation trials where we had sufficient iEEG activity and VAS scales. All stimulation was at 100 Hz, 100 us, and 1-3 mA, bipolar contacts 2+/3−. Stimulation was delivered continuously on 4/5 trials. The change was examined in relation to the change in symptom severity due to stimulation.

We validated the biomarker once the NeuroPace RNS System was in place. In the laboratory setting we streamed continuous neural data (electrocorticography) using the RNS System wand held over the cranially implanted neurostimulator with a flexible metal arm. Once final RNS detection parameters were identified, we collected 18 trials with 10 minutes of resting state neural activity followed by depression measures over the course of 7 days. As in the 10-day mapping period, the patient engaged in a range of naturalistic activities during the recordings. Logistic and linear regression models were then constructed to assess the relationship of AMY gamma power and symptom severity state or VAS-D, HAMD6 scores respectively.

### Effective Connectivity:

Effective connectivity is a functional connectivity measure that describes the directional influence of one brain region on another¹⁶. Intracranial EEG and intracortical stimulation offer the rare and unique capability to directly probe effective network connectivity through the delivery of brief stimulation pulses across adjacent contacts in one brain region, and examination of the electrical effect of perturbation—the evoked potentials (EPs)—in distant contacts. EP-mapping was performed on day 1. We delivered bipolar single pulse stimulation (1, 3, 6 mA) at 0.5 Hz for 40 s to adjacent contact pairs across a central contact pair in each brain region. Standard methods were used^{20,44,47} to measure and quantify EPs including calculating the mean across 20 trials, z-scoring voltage against 50 ms of pre-stimulus baseline, identifying N1 and N2 by their peak deflection magnitude within the 10-50 ms and 50-500 ms time-windows respectively, constructing connectivity matrices from N1 response amplitude for the full set of EPs, and Filtering on statistically significant EPs to generate a directed network graph. We determined which electrodes exhibited statistically significant EPs by comparing the distribution of N1 responses due to 20 single pulses to the distribution of pre-stimulation baseline fluctuations. The baseline score quantified the amount of spontaneous voltage fluctuation in the absence of stimulation and was calculated by finding the peak deflection magnitude during the 50 ms prior to stimulation, z-scored against the mean voltage in the 50-100 ms window prior to stimulation. We used a Wilcoxon signed-rank one-sided test with Benjamini-Hochberg false-discovery rate correction to determine if the N1 response was statistically larger than the baseline spontaneous fluctuation. Compared to a uniform z-score threshold²¹, this approach allowed us to account for variable noise between electrodes and identify true EPs with greater sensitivity.

In graph theory, a graph represents the electrophysiology network where nodes are considered to be individual electrodes and the directed link or edge between nodes represent the effect of stimulation of the source node on the amplitude of the neural signal at a distal node⁴⁸. We computed two graph theoretical metrics from the EP connectivity map—indegree and outdegree—to determine the direction and strength of functional connections between does and inform the relationship of stimulation and sensing target candidates. A node with high outdegree is a hub of high network influence in that stimulation there highly affects other nodes in the network and could be indicative of a good treatment target⁴⁹. A node with high indegree suggests a region that is influenced by stimulation in other regions and may play a role in sensing modifiable neural signatures of MDD that normalize with stimulation elsewhere in the network. We calculated indegree by taking the weighted sum of significant connections (N1 amplitudes) pointed toward each node, and outdegree by the weighted sum of significant connections projecting away from each node for right and left hemispheres and the total brain network. We identified those hubs that had the highest values and were directionally connected.

### Diffusion Tensor Imaging

Diffusion tensor imaging (DTI) can be used to map structural connectivity by identifying putative axonal fiber tracts that might mediate effective connectivity, thereby constraining our model of functional integration across distant brain regions⁴⁶. Engagement of specific white matter tracts or the intersection of several tracts has been shown to improve outcomes in deep brain stimulation for depression^{50,51}. While the exact relationship between structural and functional connectivity remains unknown, it has been suggested that structural connectivity properties can be implemented as an informative prior in a Bayesian model of effective connectivity⁵² Diffusion data was acquired using axial DTI High Angular Resolution Diffusion Imaging (HARDI) at 3Tesla with a 32-channel head coil (B-Value: 2000 s/mm², 55 directions). Tractography was performed using deterministic fiber assignment by continuous tracking (FACT)⁵³, implemented within BrainLAB FiberTracking software. Tractography was based on 3 mm diameter spherical ROIs centered on each contact of a candidate stimulation and sensing pair; DTI parameters were the same for all pairs with minimum fractional anisotropy (FA)=0.1; minimum fiber length=80 mm; maximum angulation=20 degrees. Resulting fiber tracts are color coded by orientation (red: left-right; green: anterior-posterior; blue superior-inferior). Using these parameters, the number of streamlines was used to compare the strength of connectivity for each candidate stimulation-sensing pair (VC/VS stim-AMY sens, VC/VS stim-OFC sens, SGC stim-AMY sens, and OFC Stim-AMY sens).

### Closed-Loop Control

The NeuroPace RNS System was first placed in a sensing-only configuration where neural biomarkers could be detected but no stimulation was delivered (0 mA). For the first 2.5 months, we found a detection algorithm that could identify the AMY gamma biomarker, modifying several parameters including detector number, bandpass threshold, window size, and count criterion. Our final detector included the following parameters: Pattern A1 (AMY¾): min-max frequency: 28.8-125 Hz, minimum amplitude 0.8%, window size 160 ms, count criterion 10, bandpass threshold 3, detect analysis window size 2048:Pattern A2 (AMY½): min-max frequency 28.8-125 Hz, min amplitude 0.8%, window size 160 ms, count criterion 10, bandpass threshold 10, detect analysis window size 2048. While the RNS System does not implement standard frequency decomposition algorithms, the half-wave detector approximates frequency content. In combination with the minimal amplitude settings, the detector was found to track gamma activity. With this detector ON we examined the number of detections in the 10-minute resting-state intervals for the 18 trials utilized for biomarker validation. Logistic and linear regression models were constructed using the number of stimulations as the dependent variable and the symptom state or HAMD6/VAS-D score as the independent variable. On day 74, the RNS System was turned on in closed-loop mode with stimulation ON (1 mA, 120 us, 100 Hz). We capped therapy number at 300 per day (30 min total stimulation per day) which was reached 81.1% of days. On days that the limit was reached, this occurred on average at 7:40 pm (SD 3.3 hours). This limit was selected as stimulation delivered into the evening hours interfered with the patient's sleep. Therapy limit reset time was 7:00 am.

To investigate the relationship between daily symptom severity scores (VAS-D) and the time series of the daily number of biomarker detection counts, we used dynamic time warping (DTW), a method demonstrated to be effective at relating time series exhibiting naturalistic noise⁵⁴. After normalizing symptom severity (VAS-D) and biomarker detection count time traces by their means, we computed the DTW warping function and distance²³. In order to prevent the warping function from skipping important symptom severity or biomarker features and to cohere with standard practice, we incorporated a Sakoe-Chiba warping window of size three⁵⁵. We tested the significance of the symptom severity and biomarker relationship by comparing our DTW distance to DTW distances computed between symptom severity time series and scrambled biomarker detection time series, where the biomarker time series was obtained by randomly shuffling the observed values 10,000 times.

### REFERENCES

1. Howland, R. H. Sequenced Treatment Alternatives to Relieve Depression (STAR*D). Part 2: Study outcomes. J Psychosoc Nurs Ment Health Serv 46, 21-24 (2008).
2. Holtzheimer. P. E. et al. Subcallosal cingulate deep brain stimulation for treatment-resistant depression: a multisite, randomised, sham-controlled trial. Lancet Psychiatry 4, 839-849 (2017).
3. Dougherty, D. D., et al. A Randomized Sham-Controlled Trial of Deep Brain Stimulation of the Ventral Capsule/Ventral Striatum for Chronic Treatment-Resistant Depression. Biol Psychiatry 78, 240-248 (2015).
4. Bergfeld, I. O., et al. Deep Brain Stimulation of the Ventral Anterior Limb of the Internal Capsule for Treatment-Resistant Depression: A Randomized Clinical Trial. JAMA Psychiatry 73, 456-464 (2016).
5. Weaver, F. M., et al. Randomized trial of deep brain stimulation for Parkinson disease: thirty-six-month outcomes. Neurology 79, 55-65 (2012).
6. Fisher, R., et al. Electrical stimulation of the anterior nucleus of thalamus for treatment of refractory epilepsy. Epilepsia 51, 899-908 (2010).
7. Drobisz. D. & Damborska, A. Deep brain stimulation targets for treating depression. Behav Brain Res 359, 266-273 (2019).
8. Williams, L. M. Precision psychiatry: a neural circuit taxonomy for depression and anxiety. Lancet Psychiatry 3, 472-480 (2016).
9. Bouthour, W., et al. Biomarkers for closed-loop deep brain stimulation in Parkinson disease and beyond. Nat Rev Neural 15, 343-352 (2019).
10. Scangos, K. W., Makhoul, G. S., Sugrue, L. P., Chang, E. F. & Krystal, A. D. State-dependent responses to intracranial brain stimulation in a patient with depression. Nat Med (2021).
11. Chiang, S., et al. Evidence of state-dependence in the effectiveness of responsive neurostimulation for seizure modulation. Brain Stimul 14, 366-375 (2021).
12. Fasano, A. & Helmich, R. C. Tremor habituation to deep brain stimulation: Underlying mechanisms and solutions. Mov Disord 34, 1761-1773 (2019).
13. Malone, D. A., Jr., et al. Deep brain stimulation of the ventral capsule/ventral striatum for treatment-resistant depression. Biol Psychiatry 65, 267-275 (2009).
14. Rao. V. R., et al. Direct Electrical Stimulation of Lateral Orbitofrontal Cortex Acutely Improves Mood in Individuals with Symptoms of Depression. Curr Biol 28, 3893-3902 e3894 (2018).
15. Kirkby. L. A., et al. An Amygdala-Hippocampus Subnetwork that Encodes Variation in Human Mood. Cell 175, 1688-1700 e1614 (2018).
16. Scangos, K. W., et al. Pilot Study of An Intracranial Electroencephalography Biomarker of Depressive Symptoms in Epilepsy. J. Neuropsychiatry Chin Neurosci 32, 185-190 (2020).
17. Mayberg, H. S., et al. Deep brain stimulation for treatment-resistant depression. Neuron 45, 651-660 (2005).
18. Yeung, A. W. K. & Wong, N. S. M. The Historical Roots of Visual Analog Scale in Psychology as Revealed by Reference Publication Year Spectroscopy. Front Hum Neurosci 13, 86 (2019).
19. Timmerby. N., Andersen, J. H., Sondergaard, S., Ostergaard, S. D. & Bech, P. A Systematic Review of the Clinimetric Properties of the 6-Item Version of the Hamilton Depression Rating Scale (HAM-D6). Psychother Psychoson 86, 141-149 (2017).
20. Keller, C. J., et al. Mapping human brain networks with cortico-cortical evoked potentials. Philos Trans R Soc Lond B Biol Si 369(2014).
21. Keller, C. J., et al. Corticocortical evoked potentials reveal projectors and integrators in human brain networks. J Neurosci 34, 9152-9163 (2014).
22. Sun, F. T. & Morrell, M. J. The RNS System: responsive cortical stimulation for the treatment of refractory partial epilepsy. Expert Rev Med Devices 11, 563-572 (2014).
23. Toni, G. Computing and visualizing dynamic time warping alignments in R: The dtw package. Journal of Statistical Software 31, 1-24 (2009).
24. Garcia-Lorenzo, B., et al. Stereoelectroencephalography for Refractory Epileptic Patients Considered for Surgery: Systematic Review. Meta-Analysis, and Economic Evaluation. Neurosurgery 84, 326-338 (2019).
25. Duval, E. R., Javanbakht, A. & Liberzon, 1. Neural circuits in anxiety and stress disorders: a focused review. Ther Clin Risk Manag 11, 115-126 (2015).
26. Zheng. J., et al. Amygdala-hippocampal dynamics during salient information processing. Nat Cominun 8, 14413 (2017).
27. Price, J. L. & Drevets, W. C. Neurocircuitry of mood disorders. Neuropsychopharmacology) 35, 192-216 (2010).
28. Schlaepfer, T. E., et al. Deep brain stimulation to reward circuitry alleviates anhedonia in refractory major depression. Neuropsychopharmacology 33, 368-377 (2008).
29. Lozano, A. M., et al. Subcallosal cingulate gyrus deep brain stimulation for treatment-resistant depression. Biol Psychiatry 64, 461-467 (2008).
30. Mayberg, H. S., et al. Cingulate function in depression: a potential predictor of treatment response. Neuroreport 8, 1057-1061 (1997).
31. Waltz, E. Green light for deep brain stimulator incorporating neurofeedback. Nat Biotechnol 38, 1014-1015 (2020).
32. Guinjoan, S. M., et al. Asymmetrical contribution of brain structures to treatment-resistant depression as illustrated by effects of right subgenual cingulum stimulation. J Neuropsychiatry Clin Neurosci 22, 265-277 (2010).
33. Mayberg, H. S., et al. Reciprocal limbic-cortical function and negative mood: converging PET findings in depression and normal sadness. Am J Psychiatry 156, 675-682 (1999).
34. Haq, I. U., et al. Smile and laughter induction and intraoperative predictors of response to deep brain stimulation for obsessive-compulsive disorder. Neuroimage 54 Suppl 1, S247-255 (2011).
35. Gibson, W. S., et al. The Impact of Mirth-Inducing Ventral Striatal Deep Brain Stimulation on Functional and Effective Connectivity. Cereb Cortex 27, 2183-2194 (2017),
36. Dunner, D. L. et al. Prospective, long-term, multicenter study of the naturalistic outcomes of patients with treatment-resistant depression. J Clin Psychiatry 67, 688-695 (2006).
37. Riva-Posse, P., et al. Defining critical white matter pathways mediating successful subcallosal cingulate deep brain stimulation for treatment-resistant depression. Biol Psychiatry 76, 963-969 (2014).
38. Campbell, M. C., e al. Mood response to deep brain stimulation of the subthalamic nucleus in Parkinson's disease. J Neuropsychiatry Clin Neurosci 24, 28-36 (2012).
39. Salloum, N. C. et al. Efficacy of intravenous ketamine treatment in anxious versus nonanxious unipolar treatment-resistant depression. Depress Anxiety 36, 235-243 (2019).
40. Harris, P. A., e al. The REDCap consortium: Building an international community of software platform partners. J Biomed Inform 95, 103208 (2019).
41. Harris, P. A., et al. Research electronic data capture (REDCap)—a metadata-driven methodology and workflow process for providing translational research informatics support. J Biomed Inform 42, 377-381 (2009).
42. Mukamel, R. & Fried, 1. Human intracranial recordings and cognitive neuroscience. Annu Rev Psychol 63, 511-537 (2012).
43. Chang, E. F. Towards large-scale, human-based, mesoscopic neurotechnologies. Neuron 86, 68-78 (2015).
44. Matsumoto, R., et al. Functional connectivity in the human language system: a cortico-cortical evoked potential study. Brain 127, 2316-2330 (2004).
45. Schiff. S. J., Aldroubi, A., Unser. M. & Sato, S. Fast wavelet transformation of EEG. Electroencephalogr Clin Neurophysiol 91, 442-455 (1994).
46. Friston, K. J. Functional and effective connectivity: a review. Brain Connect 1, 13-36 (2011).
47. Keller, C. J., et al. Induction and Quantification of Excitability Changes in Hunan Cortical Networks. J Neurosci 38, 5384-5398 (2018).
48. Bassett, D. S. & Sporns, O. Network neuroscience. Nat Neurosci 20, 353-364 (2017).
49. Khambhati, A. N., et al. Functional control of electrophysiological network architecture using direct neurostimulation in humans. Netw Neurosci 3, 848-877 (2019).
50. Riva-Posse, P., et al. A connectomic approach for subcallosal cingulate deep brain stimulation surgery: prospective targeting in treatment-resistant depression. Mol Psychiatry 23, 843-849 (2018).
51. Choi, K. S., Riva-Posse, P., Gross, R. E. & Mayberg, H. S. Mapping the “Depression Switch” During Intraoperative Testing of Subcallosal Cingulate Deep Brain Stimulation. JAMA Neurol 72, 1252-1260 (2015).
52. Sokolov, A. A., et al. Linking structural and effective brain connectivity: structurally informed Parametric Empirical Bayes (si-PEB). Brain Struct Funct 224, 205-217 (2019).
53. Mori, S. & van Zijl, P. C. Fiber tracking: principles and strategies—a technical review. NMR Biomed 15, 468-480 (2002).
54. Rabiner, L. R., Juang, B. H. Fundamentals of Speech Recognition, (PTR Prentice Hall. Englewood Cliffs, N. J., 1993).
55. Sakoe, H, Chiba, S. Dynamic programming algorithm optimization for spoken word recognition. IEEE Transactions on Acoustics, Speech, and Signal Processing 26, 43-49 (1978).

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims. It is also to be understood that the terminology used herein is for the purpose of describing particular embodiments only, and is not intended to be limiting, since the scope of the present invention will be limited only by the appended claims.

Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of present invention is embodied by the appended claims.

What is claimed is:

1. A closed-loop method for treating a neuropsychiatric disorder in a subject, the method comprising:
mapping a brain of a subject to identify an optimal location in a first brain region for positioning a first electrode for delivery of electrical stimulation to treat the neuropsychiatric disorder;
mapping the brain of the subject to identify an optimal location in a second brain region for positioning a second electrode for detection of an electroencephalographic signal from neural activity associated with a symptom of the neuropsychiatric disorder;
positioning the first electrode at the optimal location in the first brain region to deliver electrical stimulation to the first brain region of the subject;
positioning the second electrode at the optimal location in the second brain region to detect the electroencephalographic signal from the neural activity associated with the symptom of the neuropsychiatric disorder;
detecting the electroencephalographic signal at the second region of the brain of the subject using the second electrode; and
applying electrical stimulation to the first brain region using the first electrode in a manner effective to treat the neuropsychiatric disorder in the subject when the electroencephalographic signal that is detected using the second electrode exceeds a threshold level.

2. The method of claim 1, further comprising using a control algorithm to automate said applying electrical stimulation when the electroencephalographic signal exceeds the threshold level.

3. The method of claim 2, wherein the control algorithm comprises a machine learning algorithm.

4. The method of claim 3, wherein the machine learning algorithm is a supervised machine learning algorithm.

5. The method of any one of claims 2-4, wherein the control algorithm further modulates one or more programmed stimulation parameters based on the level of the electroencephalographic signal.

6. The method of any one of claims 1-5, further comprising positioning a plurality of electrodes at the second brain region for detection of the electroencephalographic signal by stereoelectroencephalography (sEEG).

7. The method of any one of claims 1-6, wherein the first brain region comprises a ventral capsule/ventral striatum (VC/VS) region, a subgenual cingulate (SGC) region, or an orbitofrontal cortex (OFC) region of the brain.

8. The method of any one of claims 1-7, wherein the second brain region comprises a right amygdala region, a left amygdala region, a right orbitofrontal cortex region, a left subgenual cingulate region, or a right hippocampus region.

9. The method of any one of claims 1-8, wherein the electroencephalographic signal is gamma frequency power.

10. The method of any one of claims 1-9, wherein the first electrode is placed on a surface of the first brain region or within the first brain region.

11. The method of any one of claims 1-10, wherein the second electrode is placed on a surface of the second brain region or within the second brain region.

12. The method of any one of claims 1-11, wherein the first electrode or the second electrode is a non-brain penetrating surface electrode array.

13. The method of any one of claims 1-12, wherein the first electrode or the second electrode is a brain-penetrating electrode array.

14. The method of any one of claims 1-13, wherein the neuropsychiatric disorder comprises depression, wherein applying the electrical stimulation treats depression.

15. The method of any one of claims 1-14, wherein the neuropsychiatric disorder comprises anxiety, wherein applying the electrical stimulation treats anxiety.

16. The method of any one of claims 1-15, wherein the neuropsychiatric disorder is Major Depressive Disorder (MDD), Generalized Anxiety Disorder (GAD), Post-Traumatic Stress Disorder (PTSD), Addiction, Anorexia, Obsessive-Compulsive Disorder (OCD), or Bipolar Disorder (BD) or chronic pain.

17. The method of any one of claims 1-16, wherein the electrical stimulation is applied unilaterally or bilaterally.

18. The method of any one of claims 1-17, wherein the method further comprises assessing effectiveness of the treatment in the subject.

19. The method of any one of claims 1-18, further comprising monitoring the subject for a period of time, wherein increasing numbers of the electroencephalographic signals exceeding the threshold level detected by the second electrode within a later recording period compared to an earlier recording period indicate increasing severity of the symptom of the neuropsychiatric disorder; and decreasing numbers of electroencephalographic signals exceeding the threshold level detected by the second electrode within a later recording period compared to an earlier recording period indicate decreasing severity of the symptom of the neuropsychiatric disorder.

20. The method of claim 19, wherein the subject is monitored continuously or intermittently.

21. The method of any one of claims 1-20, wherein the first brain region and the second brain region are the same or different.

22. A closed-loop method for treating major depressive disorder (MDD) in a subject, the method comprising:
positioning a first electrode to deliver electrical stimulation to a first region of the brain of the subject;
positioning a second electrode to detect a gamma frequency electroencephalographic signal from neural activity associated with MDD symptoms at a second region of the brain of the subject;
detecting the gamma frequency electroencephalographic signal using the second electrode; and
applying electrical stimulation to the first region of the brain of the subject using the first electrode in a manner effective to treat the MDD symptoms in the subject when the gamma frequency electroencephalographic signal that is detected using the second electrode exceeds a threshold level.

23. The method of claim 22, wherein the first brain region comprises a ventral capsule/ventral striatum (VC/VS) region, a subgenual cingulate (SGC) region, or an orbitofrontal cortex (OFC) region of the brain.

24. The method of claim 22 or 23, wherein the second brain region comprises a right amygdala, left amygdala region, a right orbitofrontal cortex region, a left subgenual cingulate region, or a right hippocampus region.

25. The method of claim 23 or 24, wherein said applying electrical stimulation comprises applying bipolar electrical stimulation at the VC/VS region of the brain of the subject.

26. The method of any one of claims 22-25, wherein said electrical stimulation is applied at a current ranging from 1 mA to 6 mA.

27. The method of any one of claims 22-26, wherein said electrical stimulation is applied for up to 10 minutes a day.

28. The method of any one of claims 22-26, wherein said electrical stimulation is applied continuously for a time ranging from 1 hour a day to 24 hours a day.

29. The method of any one of claims 22-28, further comprising monitoring the subject for a period of time, wherein increasing numbers of gamma frequency electroencephalographic signals exceeding the threshold level detected by the second electrode within a later recording period compared to an earlier recording period indicate increasing severity of MDD symptoms; and decreasing numbers of gamma frequency electroencephalographic signals exceeding the threshold level detected by the second electrode within a later recording period compared to an earlier recording period indicate decreasing severity of MDD symptoms.

30. The method of claim 29, wherein the subject is monitored continuously or intermittently.

31. A closed-loop method for ameliorating a symptom of a neuropsychiatric disorder in a subject, the method comprising:
mapping a brain of a subject to identify an optimal location in a first brain region for positioning a first electrode for delivery of electrical stimulation to ameliorate the symptom of the neuropsychiatric disorder;
mapping the brain of the subject to identify an optimal location in a second brain region for positioning a second electrode for detection of an electroencephalographic signal from neural activity associated with the symptom of the neuropsychiatric disorder;
positioning the first electrode at the optimal location in the first brain region to deliver electrical stimulation to the first brain region of the subject;
positioning the second electrode at the optimal location in the second brain region to detect the electroencephalographic signal from the neural activity associated with the symptom of the neuropsychiatric disorder;
detecting the electroencephalographic signal at the second region of the brain of the subject using the second electrode; and
applying electrical stimulation to the first brain region using the first electrode in a manner effective to ameliorate the symptom of the neuropsychiatric disorder in the subject when the electroencephalographic signal that is detected using the second electrode exceeds a threshold level.

32. The method of claim 31, wherein the symptom comprises depression and wherein applying the electrical stimulation ameliorates depression.

33. The method of claim 32, wherein depression is measured by Visual Analogue Scale for Depression (VAS-D), Hamilton Depression Rating Scale (HAM-D), Montgomery-Asberg Depression Rating Scale (MADRS), or Beck Depression Inventories (BDI) score and applying the electrical stimulation is effective in reducing symptom severity according to the VAS, HAM-D, MADRS, or BDI score.

34. The method of claim 31, wherein the symptom comprises anxiety and wherein applying the electrical stimulation ameliorates anxiety.

35. The method of claim 34, wherein anxiety is measured by Visual Analogue Scale for Anxiety (VAS-A) or Beck Anxiety Inventories (BAI) score and applying the electrical stimulation is effective in reducing anxiety according to the VAS-A or the BAI score.

36. The method of any one of claims 31-35, wherein the first electrode is placed on a surface of the first brain region or within the first brain region.

37. The method of any one of claims 31-36, wherein the second electrode is placed on a surface of the second brain region or within the second brain region.

38. The method of any one of claims 31-37, wherein the first electrode or the second electrode is a non-brain penetrating surface electrode array.

39. The method of any one of claims 31-38, wherein the first electrode or the second electrode is a brain-penetrating electrode array.

40. The method of any one of claims 31-39, wherein the neuropsychiatric disorder is Major Depressive Disorder (MDD), Generalized Anxiety Disorder (GAD), Post-Traumatic Stress Disorder (PTSD), addiction, anorexia, Obsessive-Compulsive Disorder (OCD), or Bipolar Disorder (BD), or chronic pain.

41. The method of any one of claims 31-40, wherein the method further comprises assessing effectiveness of the treatment in the subject.

42. The method of any one of claims 31-41, further comprising using a control algorithm to automate said applying electrical stimulation when the electroencephalographic signal exceeds the threshold level.

43. The method of claim 42, wherein the control algorithm comprises a machine learning algorithm.

44. The method of claim 43, wherein the machine learning algorithm is a supervised machine learning algorithm.

45. The method of any one of claims 42-44, wherein the control algorithm further modulates one or more programmed stimulation parameters based on the level of the electroencephalographic signal.

46. The method of any one of claims 31-45, further comprising positioning a plurality of electrodes at the second brain region for detection of the electroencephalographic signal by stereoelectroencephalography (sEEG).

47. The method of any one of claims 31-46, wherein the first brain region comprises a ventral capsule/ventral striatum (VC/VS) region, a subgenual cingulate (SGC) region, or an orbitofrontal cortex (OFC) region of the brain.

48. The method of any one of claims 31-47, wherein the second brain region comprises a right amygdala region, a left amygdala region, a right orbitofrontal cortex region, a left subgenual cingulate region, or a right hippocampus region.

49. The method of any one of claims 31-48, wherein the electroencephalographic signal is gamma frequency power.

50. A system for treating a neuropsychiatric disorder in a subject, the system comprising:
a stimulation electrode adapted for positioning at a first region of the brain of the subject;
a detection electrode adapted for positioning at a second region of the brain of the subject and for recording an electroencephalographic signal from the second region of the brain of the subject; and
a processor programmed to instruct the stimulation electrode to apply an electrical stimulation to the first region of the brain in a manner effective to treat the neuropsychiatric disorder in the subject when an electroencephalographic signal that exceeds a threshold level is detected using the second electrode.

51. The system of claim 50, wherein the first brain region comprises a ventral capsule/ventral striatum (VC/VS) region, a subgenual cingulate (SGC) region, or an orbitofrontal cortex (OFC) region of the brain.

52. The system of claim 50 or 51, wherein the second brain region comprises a right amygdala region, a left amygdala region, a right orbitofrontal cortex region, a left subgenual cingulate region, or a right hippocampus region.

53. The system of any one of claims 50-52, wherein the electroencephalographic signal is gamma frequency power.

54. The system of any one of claims 50-53, wherein the stimulation electrode or the detection electrode is a non-brain penetrating electrode array.

55. The system of any one of claims 50-54, wherein the stimulation electrode or the detection electrode is a brain penetrating electrode array.

56. The system of any one of claims 50-55, wherein the neuropsychiatric disorder comprises depression, wherein applying electrical stimulation treats the depression.

57. The system of any one of claims 50-56, wherein the neuropsychiatric disorder comprises anxiety, wherein applying electrical stimulation treats the anxiety.

58. The system of any one of claims 50-57, wherein the system further comprises a user interface comprising an input electronically coupled to the processor for instructing the stimulation electrode to apply an electrical stimulation to the first region of the brain to treat the neuropsychiatric disorder in the subject.

59. The system of claim 58, wherein the user interface is password protected and is operable by a health care practitioner.

60. The system of any one of claims 50-59, wherein the neuropsychiatric disorder is Major Depressive Disorder (MDD), Generalized Anxiety Disorder (GAD), Post-Traumatic Stress Disorder (PTSD), Addiction, Anorexia, Obsessive-Compulsive Disorder (OCD), or Bipolar Disorder (BD), or chronic pain.

61. The system of any one of claims 50-60, wherein the processor is further programmed to modulate one or more programmed stimulation parameters according to the algorithm's control law; and apply the modulated electrical stimulation to the first region of the brain via the stimulation electrode in a manner effective to treat the neuropsychiatric disorder.

62. The system of any one of claims 50-61, wherein the processor is further programmed to set a maximum number of electrical stimulations per day.

63. The system of any one of claims 50-62, wherein the processor is further programmed to set a total amount of time of electrical stimulation per day.

64. A system for ameliorating a symptom of a neuropsychiatric disorder in a subject, the system comprising:
a stimulation electrode adapted for positioning at a first region of the brain of the subject;
a detection electrode adapted for positioning at a second region of the brain of the subject and for recording an electroencephalographic signal from the second region of the brain of the subject; and
a processor programmed to instruct the stimulation electrode to apply an electrical stimulation to the first region of the brain in a manner effective to ameliorating the symptom of the neuropsychiatric disorder in the subject when an electroencephalographic signal that exceeds a threshold level is detected using the detection electrode.

65. The system of claim 64, wherein the first brain region comprises a ventral capsule/ventral striatum (VC/VS) region, a subgenual cingulate (SGC) region, or an orbitofrontal cortex (OFC) region of the brain.

66. The system of claim 64 or 65, wherein the second brain region comprises a right amygdala region, a left amygdala region, a right orbitofrontal cortex region, a left subgenual cingulate region, or a right hippocampus region.

67. The system of any one of claims 64-66, wherein the electroencephalographic signal is gamma frequency power.

68. The system of any one of claims 64-67, wherein the symptom comprises depression and wherein applying the electrical stimulation ameliorates depression.

69. The system of any one of claims 64-68, wherein the symptom comprises anxiety and wherein applying the electrical stimulation ameliorates anxiety.

70. The system of any one of claims 64-69, wherein the stimulation electrode or the detection electrode is a non-brain penetrating electrode.

71. The system of any one of claims 64-70, wherein the stimulation electrode or the detection electrode is a brain penetrating electrode.

72. The system of any one of claims 64-71, wherein the system further comprises a user interface comprising an input electronically coupled to the processor for instructing the stimulation electrode to apply an electrical stimulation to the first region to ameliorate the symptom of the neuropsychiatric disorder in the subject.

73. The system of claim 72, wherein the user interface is password protected and is operable by a health care practitioner.

74. The system of any one of claims 64-73, wherein the neuropsychiatric disorder is Major Depressive Disorder (MDD), Generalized Anxiety Disorder (GAD), Post-Traumatic Stress Disorder (PTSD), Addiction, Anorexia, Obsessive-Compulsive Disorder (OCD), or Bipolar Disorder (BD), or chronic pain.

75. The system of any one of claims 64-74, wherein the processor is further programmed to modulate one or more programmed stimulation parameters according to the algorithm's control law; and apply the modulated electrical stimulation to the first region of the brain via the stimulation electrode in a manner effective to ameliorate the symptom of the neuropsychiatric disorder.

76. The system of any one of claims 64-75, wherein the processor is further programmed to set a maximum number of electrical stimulations per day.

77. The system of any one of claims 64-76, wherein the processor is further programmed to set a total amount of time of electrical stimulation per day.
